(19) United States
(12) Patent Application Publication
(10) Pub. No.: US 2023/0106770 A1
(43) Pub. Date: Apr. 6, 2023

(54) Reagents and methods for preventing, treating or limiting severe acute respiratory syndrome (SARS) coronavirus infection

(71) Applicant: PHYLEX BIOSCIENCES, INC., Del Mar, CA (US)

(72) Inventor: Pascal BRANDYS, Del Mar, CA (US)

(21) Appl. No.: 17/905,315

(22) PCT Filed: Apr. 2, 2021
(86) PCT No.: PCT/US2021/025508

§ 371 Date: Aug. 30, 2022

Related U.S. Application Data
(60) Provisional application No. 63/006,068, filed on Apr. 6, 2020.
(60) Provisional application No. 63/011,834, filed on Apr. 17, 2020.
(60) Provisional application No. 63/048,102, filed on Jul. 4, 2020.
(60) Provisional application No. 63/048,458, filed on Jul. 6, 2020.

(51) Int. Cl. A61K 39/215; A61P 31/14

(52) U.S. Cl. CPC A61K 39/215; A61P 31/14; A61K 2039/53

(57) ABSTRACT

Isolated antigenic polypeptides, multimers thereof, encoding nucleic acids, and pharmaceutical compositions are provided that can be used for treating or limiting development of a sever acute respiratory (SARS) coronavirus infection.

### CROSS REFERENCE

This application claims priority to U.S. Provisional Patent Application Serial Nos. 63/006,068 filed Apr. 6, 2020; 63/011,834 filed Apr. 17, 2020; 63/048,102 filed Jul. 4, 2020; and 63/048,458 filed Jul. 6, 2020, each incorporated by reference herein in its entirety

### SEQUENCE LISTING STATEMENT

A computer readable form of the Sequence Listing is filed with this application by electronic submission and is incorporated into this application by reference in its entirety. The Sequence Listing is contained in the file created on Mar. 17, 2021, having the file name “20-994-WO_SeqList_ST25.txt” and is 41 KB in size.

### BACKGROUND

Three highly pathogenic human coronaviruses (CoVs) have been identified to date: severe acute respiratory syndrome (SARS) coronavirus (SARS-CoV), Middle East 20 respiratory syndrome coronavirus (MERS-CoV) and a 2019 novel coronavirus (2019-nCoV), as previously termed by the World Health Organization (WHO).

Severe acute respiratory syndrome (SARS) caused by the novel human coronavirus SARS-CoV emerged from Guangdong Province, China, in late 2002. By the end of 2003, it had spread to more than 30 countries, affecting 8,096 people and causing 774 deaths, with a 25 case fatality rate (CFR) of about 10%. Although the global SARS pandemic was brought under control in July 2003, reports of sporadic cases in China from late 2003 to early 2004 raised concerns about the reemergence of SARS-CoV through zoonotic reintroduction.

Middle East respiratory syndrome (MERS) caused by MERS-CoV, a close relative to SARS-CoV, was reported in Saudi Arabia in June 2012. As compared to SARS-CoV, MERS-CoV showed limited human-to-human transmission but a higher CFR of about 35%.

The 2019-nCoV was first reported in Wuhan, China in December 2019 from patients with pneumonia, and it has exceeded both SARS-CoV and MERS-CoV in its rate of transmission among humans. 2019-nCoV was renamed SARS-CoV-2 by Coronaviridae Study Group (CSG) of the International Committee on Taxonomy of Viruses (ICTV). The disease and the virus causing it were named Coronavirus Disease 2019 (COVID-19) and the COVID-19 virus, respectively, by the WHO. As of Jul. 2, 2020, more than 10.6 million cases of COVID-19 were reported, resulting in more than 516,000 deaths, in at least 200 countries and territories. Currently, the intermediate host of SARS-CoV-2 is still unknown, and no effective prophylactics or therapeutics are available.

SARS-CoV is a single, non-segment and positive-stranded RNA virus with envelope. Its genomic RNA consists of 29,736 nucleotides, two thirds of its 5-encoding nonstructural RNA replicase polyprotein and one third of its 3′-encoding structural proteins, including spike (S), envelope (E), membrane (M), and nucleocapsid (N) proteins. SARS-CoV-2 genomic RNA consists of 29,903 nucleotides, with a similar encoding of nonstructural and structural proteins.

The SARS-CoV and SARS-CoV-2 S protein is a type I transmembrane envelope glycoprotein and consists of S1 surface subunit, which is responsible for receptor binding, and S2 transmembrane subunit, which mediates membrane fusion.

The S protein mediates viral entry into host cells by first binding to a host receptor through the receptor-binding domain (RBD) in the S1 subunit and then fusing the viral and host membranes through the S2 subunit. The entry of SARS-CoV and SARS-CoV-2 is initiated by binding of the S protein to the cellular receptor angiotensin-converting enzyme 2 (ACE2).

In SARS-CoV a fragment of 193 residues spanning the residues 318-510 in S1 subunit is the minimal RBD.

In SARS-CoV-2 the RBD is identified by alignment of RBD sequences of SARS-CoV and SARS-CoV-2 as shown in FIG. 1. SARS-CoV-2 RBD is a fragment of 194 residues, spanning the residues 331-524, with the insertion of one glutamine in position 482.

In SARS-CoV the RBD contains a loop region of 71 residues spanning residues 424-494, termed receptor-binding motif (RBM), which makes complete contact with the receptor ACE2. In SARS-CoV-2 the RBM contains 72 residues and is spanning residues 437-508 with the insertion of Gln482.

### SUMMARY

In one aspect, the disclosure provides isolated polypeptides comprising a conserved receptor-binding domain (CRBD) from a severe acute respiratory syndrome (SARS) coronavirus spike protein, wherein the CRBD comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-9, wherein the polypeptide includes no more than 10, 9, 8, 7, 6, 5, 4, 3, 2, 1 amino acid residues from a variable receptor binding motif (VRBM) from a SARS coronavirus spike protein, or includes no amino acid residues from the VRBM of a SARS coronavirus spike protein. In one embodiment, the CRBD comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-8 and 38. In another embodiment, the CRBD comprises an amino acid sequence at least 70%, 75%, 80%, 85%, 90%, 91%, 92%, 93%, 94%, 95%, 96%, 97%, 98%, 99%, or 100% identical to the amino acid sequence of SEQ ID NO:10 or 22. In a further embodiment, the CRBD comprises the amino acid sequence of SEQ ID NO:2, SEQ ID NO: 10, or SEQ ID NO:22. In another embodiment, the CRBD comprises an amino acid sequence at least 70%, 75%, 80%, 85%, 90%, 91%, 92%, 93%, 94%, 95%, 96%, 97%, 98%, 99%, or 100% identical to SEQ ID NO:9. In one embodiment, the CRBD comprises the amino acid sequence of SEQ ID NO:9 or SEQ ID NO:25.

In another embodiment, the isolated polypeptides further comprise a multimerization domain. In one embodiment, the multimerization domain comprises an amino acid sequence at least at least 70%, 75%, 80%, 85%, 90%, 91%, 92%, 93%, 94%, 95%, 96%, 97%, 98%, 99%, or 100% identical to the amino acid sequence of SEQ ID NO: 23, 24, 27, or 28. In another embodiment, the isolated polypeptides comprise the amino acid sequence selected from the group consisting of SEQ ID NOS: 11-18 and 39, wherein n is 3-7, 3-6, 3-5, 3-4, 4-7, 4-6, 4-5, 5-7, 5-6, 3, 4, 5, 6, or 7. In another embodiment, the isolated polypeptide comprises the amino acid sequence of SEQ ID NO:20, or SEQ ID NO:21, or SEQ ID NO:22. In a further embodiment, the isolated polypeptide comprises a general formula selected from the group consisting of:

X1-(GGS)nGGG-X3 and X1-(GGS)nGGG-X3-(GGS)nGGG-X2, wherein

X1 and X2 independently comprise the amino acid sequence selected from the group consisting of SEQ ID NOS: 1-22, and 25:

n is 3-5, 3-4, 4-5, 3, 4, 5 and

X3 comprises the amino acid sequence selected from the group consisting of SEQ ID NO: 27 or 28.

In one embodiment, X1 and X2 (when present) independently comprise the amino acid sequence selected from the group consisting of SEQ ID NOS: 1-8, 10, 22, and 38.

In a further embodiment, the isolated polypeptides include no amino acid residues from a SARS coronavirus spike protein VRBM.

In a further embodiment, the disclosure provides multimers comprising two or more copies of the isolated polypeptides of any embodiment or combination of embodiments disclosed herein. In one embodiment, the multimers comprise between 2 and 60 copies of the isolated polypeptides.

In another embodiment, the disclosure provides scaffolds, comprising two or more isolated polypeptides of any embodiment or combination of embodiments disclosed herein on a surface of the scaffold. In different embodiments, the two or more isolated polypeptides are all identical polypeptides, or the two or more isolated polypeptides include different polypeptides.

In one embodiment, the different polypeptides include:

(a) the CRBD of SEQ ID NO:3 and the CRBD of SEQ ID NO:4;

(b) the isolated polypeptide of SEQ ID NO:13 and the isolated polypeptide of SEQ ID NO:14:

(c) the CRBD of SEQ ID NO:5 and the CRBD of SEQ ID NO:6;

(d) the isolated polypeptide of SEQ ID NO:15 and the isolated polypeptide of SEQ ID NO:16; and/or

(e) two or all three of the isolated polypeptide of SEQ ID NO:20, the isolated polypeptide of SEQ ID NO:21, and the isolated polypeptide of SEQ ID NO:32 the isolated polypeptide of SEQ ID NO:20 and the isolated polypeptide of SEQ ID NO:21.

In another aspect, the disclosure provides nucleic acids encoding the isolated polypeptides of any embodiment or combination of embodiments disclosed herein. In one embodiment, the nucleic acid comprises mRNA. In other embodiments, the mRNA comprises a 5′ cap, a poly(A) tail of between 50 and 120 contiguous adenosine residues, a 5′ untranslated region comprising the sequence GGGAGACUGCCACCAUG (SEQ ID NO:33) or GGGAGACUGCCAAGAUG (SEQ ID NO:34), and/or a Y untranslated region comprising one or two copies of a beta globin mRNA 3′-UTR. In other aspects, the disclosure provides recombinant expression vector comprising the nucleic acid of the disclosure operatively linked to a suitable control sequence, and recombinant host cells comprising the polypeptide, the multimer, the scaffold, the nucleic acid, and/or the recombinant expression vector of any embodiment or combination of embodiments herein.

In another aspect, the disclosure provides pharmaceutical compositions comprising

(a) the polypeptide, the multimer, the scaffold, the nucleic acid, the recombinant expression vector, and/or the cell of any embodiment or combination of embodiments herein; and

(b) a pharmaceutically acceptable carrier.

In one embodiment, the pharmaceutical composition comprises

(a) the CRBD of SEQ ID NO:3 and the CRBD of SEQ ID NO:4;

(b) the isolated polypeptide of SEQ ID NO:13 and the isolated polypeptide of SEQ ID NO:14:

(c) the CRBD of SEQ ID NO:5 and the CRBD of SEQ ID NO:6;

(d) the isolated polypeptide of SEQ ID NO:15 and the isolated polypeptide of SEQ ID NO:16; and/or

(e) two or all three of the isolated polypeptide of SEQ ID NO:20, the isolated polypeptide of SEQ ID NO:21, and the isolated polypeptide of SEQ ID NO:32 the isolated polypeptide of SEQ ID NO:20 and the isolated polypeptide of SEQ ID NO:21:

or multimers or scaffolds thereof.

In another embodiment, the pharmaceutical composition comprises:

(a) a mRNA of any embodiment or combination of embodiments herein; and

(b) a cationic lipid such as a liposome, or a cationic protein such as protamine.

In one embodiment, the mRNA encodes:

(a) the CRBD of SEQ ID NO:3 and the CRBD of SEQ ID NO:4;

(b) the isolated polypeptide of SEQ ID NO:13 and the isolated polypeptide of SEQ ID NO:14:

(c) the CRBD of SEQ ID NO:5 and the CRBD of SEQ ID NO:6;

(d) the isolated polypeptide of SEQ ID NO:15 and the isolated polypeptide of SEQ ID NO:16; and/or

(e) two or all three of the isolated polypeptide of SEQ ID NO:20, the isolated polypeptide of SEQ ID NO:21, and the isolated polypeptide of SEQ ID NO:32 the isolated polypeptide of SEQ ID NO:20 and the isolated polypeptide of SEQ ID NO:21.

In other aspects, the disclosure provides methods for treating or limiting a severe acute respiratory (SARS) coronavirus infection, comprising administering to a subject infected with a SARS coronavirus or at risk of SARS coronavirus infection an amount effective to treat or limit development of the infection of the polypeptide, the multimer, the scaffold, the nucleic acid, the recombinant expression vector, the cell, and/or the pharmaceutical composition of any embodiment or combination of embodiments herein.

In another aspect, the disclosure provides method for generating an immune response in a subject, comprising administering to the subject an amount effective to generate an immune response of the polypeptide, the multimer, the scaffold, the nucleic acid, the recombinant expression vector, the cell, and/or the pharmaceutical composition of any embodiment or combination of embodiments herein.

In a further aspect, the disclosure provides methods for monitoring a SARS coronavirus-induced disease in a subject and/or monitoring response of the subject to immunization by a SARS coronavirus vaccine, comprising contacting the polypeptide, the multimer, the scaffold, the nucleic acid, the recombinant expression vector, the cell, and/or the pharmaceutical composition of any embodiment or combination of embodiments herein with a bodily fluid from the subject and detecting SARS coronavirus-binding antibodies in the bodily fluid of the subject.

In one aspect, the disclosure provides methods for detecting SARS coronavirus binding antibodies, comprising

(a) contacting the polypeptide, the multimer, the scaffold, and/or the pharmaceutical composition of any embodiment or combination of embodiments herein with a composition comprising a candidate SARS coronavirus binding antibody under conditions suitable for binding of SARS coronavirus antibodies to the polypeptide, the multimer, the scaffold, and/or the pharmaceutical composition; and

(b) detecting SARS coronavirus antibody complexes with the polypeptide, the multimer, the scaffold, and/or the pharmaceutical composition.

In a further aspect, the disclosure provides methods for producing SARS coronavirus antibodies, comprising

(a) administering to a subject an amount effective to generate an antibody response of the polypeptide, the multimer, the scaffold, the nucleic acid, the recombinant expression vector, the cell, and/or the pharmaceutical composition of any embodiment or combination of embodiments herein; and

(b) isolating antibodies produced by the subject.

### DESCRIPTION OF THE FIGURES

FIG. 1. Alignment of the amino acid sequence of the receptor-binding domain (RBD) of the S protein for the SARS-CoV-2 and the SARS-CoV viruses.

FIG. 2. Alignment of the amino acid sequences of the conserved receptor-binding domain (CRBD) of the S protein for the SARS-CoV-2 and SARS-CoV viruses

FIG. 3. Alignment of the amino acid sequence of the first fragment of the conserved receptor-binding domain (CRBD-1) of the S protein for SARS-CoV-2, the bat RaTG13 virus, the pangolin betacoronavirus of 2019, the pangolin betacoronavirus of 2017 and SARS-CoV.

FIG. 4. Alignment of the amino acid sequences of the first fragment of the conserved receptor-binding domain (CRBD-1) of the S protein for SARS-CoV-2 and SARS-CoV-2 including all reported recent mutations not appearing in FIG. 3.

FIG. 5. Alignment of the amino acid sequences of the first fragment of the conserved receptor-binding domain (CRBD-1) of the S protein for SARS-CoV-2 and SARS-CoV and the bat betacoronaviruses RaTG13 of 2013 and Rhinolophus affinis-CoV of 2014.

FIG. 6. Negative stain electron microscopy (NS-EM) images of exemplary VX2024 nanoparticle.

FIG. 7. Two dimensional alignment of 186 micrographs of VX2024 nanoparticle into 20 classes.

FIG. 8. Graph showing inhibition of RBD-ACE2 interaction with VX2024 vaccine in 10 mice.

FIG. 9. Graph showing inhibition of RBD-ACE2 interaction with VX2024r vaccine in 8 mice.

FIG. 10. Graph showing inhibition of RBD-ACE2 interaction with VX2024r vaccine in 8 mice and with 2 injection routes.

FIG. 11. Graph showing inhibition of RBD-ACE2 interaction with VX2024rM vaccine in 8 mice and with 2 injection routes.

### DETAILED DESCRIPTION

All references cited are herein incorporated by reference in their entirety. As used herein, the singular forms “a”, “an” and “the” include plural referents unless the context clearly dictates otherwise.

All embodiments of any aspect of the disclosure can be used in combination, unless the context clearly dictates otherwise.

As used herein, “about” means +/−5% of the recited parameter.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used throughout the present application, the terms “protein” or “polypeptide” are used in their broadest sense to refer to a sequence of subunit amino acids. The proteins or polypeptides of the disclosure may comprise L-amino acids, D-amino acids (which are resistant to L-amino acid-specific proteases in vivo), or a combination of D- and L-amino acids. The proteins or polypeptides described herein may be chemically synthesized or recombinantly expressed.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

As used throughout the present application, the term “SARS coronavirus” is used in its broadest sense to designate any highly pathogenic coronavirus phylogenetically related to SARS-CoV or SARS-CoV-2.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Parentheses represent variable positions in the polypeptides, with the recited amino acid residues within parentheses as alternatives in these positions.

An abbreviated amino acid residue preceded or followed by a number indicates the position of the amino acid in a sequence of residues.

In a first aspect, the disclosure provides isolated polypeptides comprising a conserved receptor-binding domain (CRBD) from a severe acute respiratory syndrome (SARS) coronavirus spike protein, wherein the CRBD comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-9, wherein the polypeptide includes no more than 10, 9, 8, 7, 6, 5, 4, 3, 2, or 1 amino acid residue from a variable receptor binding motif (VRBM) from a SARS coronavirus spike protein. In each sequence, residues in parentheses separated by slashes are alternative possible residues at a given position.

(SEQ ID NO: 1)
NITNLCPFGE(V/I)FN(A/S)(T/S)(R/T/K)FA
SVYAW(N/D)RKRISNCVA(D/Y)YS(V/F)LYNS
(A/T)SFSTF(K/R)CYGVSPTKLNDLCFTNVYADSF
V(I/V)(R/T/K)GDEVR(Q/E)IAPGQTG(K/R/V)
IADYNYKLPDDFTGCVI(A/S)WN
(SEQ ID NO: 2)
NITNLCPFGEVFNA(T/S)(R/T/K)FASVYAWNRK
RISNCVADYSVLYNS(A/T)SFSTFKCYGVSPTKLN
DLCFTNVYADSFV(I/V)(R/T/K)GDEVRQIAPGQ
TG(K/R/V)IADYNYKLPDDFTGCVIAWN
(SEQ ID NO: 3)
NITNLCPFGE(V/I)FN(A/S)(T/S)(R/T/K)F(
A/P)SVYAW(N/E/D)RK(R/K)ISNCVA(D/Y)YS
(V/F)LYNSA(S/F)FSTF(K/R)CYGVS(P/A)TK
LNDLCF(T/S)NVYADSFV(I/V)(R/T/K)GD(E/
D)VR(Q/E)IAPGQTG(K/R/V)IADYNYKLPDDF(
T/M)GCV(I/L)(A/S)WN
(SEQ ID NO: 4)
NITNLCPFGE(V/I)FN(A/S)(T/S)(R/T/K)F(
A/P)SVYAW(N/E/D)RK(R/K)ISNCVA(D/Y)YS
(V/F)LYNST(S/F)FSTF(K/R)CYGVS(P/A)TK
LNDLCF(T/S)NVYADSFV(I/V)(R/T/K)GD(E/
D)VR(Q/E)IAPGQTG(K/R/V)IADYNYKLPDDF(
T/M)GCV(I/L)(A/S)WN
(SEQ ID NO: 5)
NITNLCPFGEVFNA(T/S)(R/T/K)FASVYAWNRK
RISNCVADYSVLYNSASFSTFKCYGVSPTKLNDLCF
TNVYADSFV(I/V)(R/T/K)GDEVRQIAPGQTG(K
/R/V)IADYNYKLPDDFTGCVIAWN
(SEQ ID NO: 6)
NITNLCPFGEVFNA(T/S)(R/T/K)FASVYAWNRK
RISNCVADYSVLYNSTSFSTFKCYGVSPTKLNDLCF
TNVYADSFV(I/V)(R/T/K)GDEVRQIAPGQTG(K
/R/V)IADYNYKLPDDFTGCVIAWN
(SEQ ID NO: 7)
NITNLCPFGEVFNAT(R/K)F(A/P)SVYAW(N/E)
RK(R/K)ISNCVADYSVLYNS(A/T)(S/F)FSTFK
CYGVS(P/A)TKLNDLCF(T/S)NVYADSFV(I/V)
(R/K)GD(E/D)VRQIAPGQTG(K/V)IADYNYKLP
DDF(T/M)GCV(I/L)AWN
(SEQ ID NO: 8)
NITNLCPFGE(V/I)FN(A/S)(T/S)(R/T/K)F(
A/P)SVYAW(N/E/D)RK(R/K)ISNCVA(D/Y)YS
(V/F)LYNS(A/T)(S/F)FSTF(K/R)CYGVS(P/
A)TKLNDLCF(T/S)NVYADSFV(I/V)(R/T/K)G
D(E/D)VR(Q/E)IAPGQTG(K/R/V)IADYNYKLP
DDE(T/M)GCV(I/L)(A/S)WN
(SEQ ID NO: 38)
NITNLCPFGE(V/I)FN(A/S)(T/S)(R/T/K)FA
SVYAW(N/D)RKRISNCVA(D/Y)YS(V/F)LYNS(
A/T)SFSTF(K/R)CYGVSPTKLNDL(C/A)FTNVY
ADSFV(I/V)(R/T/K)GDEVR(Q/E)IAPGQTG(K
/R/V/N/T)IADYNYKLPDDFTGCVI(A/S)WN
(SEQ ID NO: 9)
GYQPYRVWLSFELL(H/N)APATV
(CRBD-2)

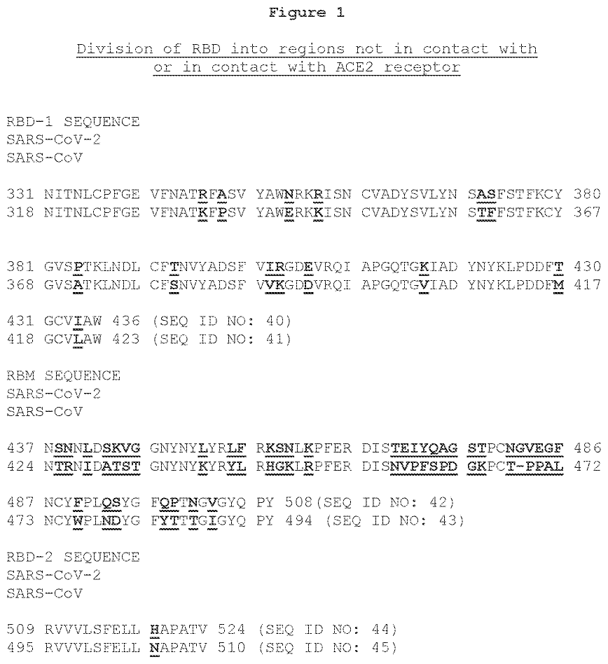

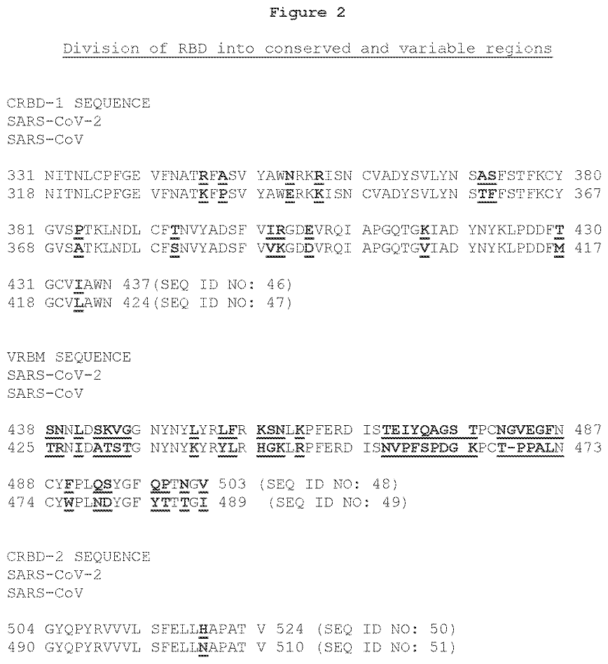

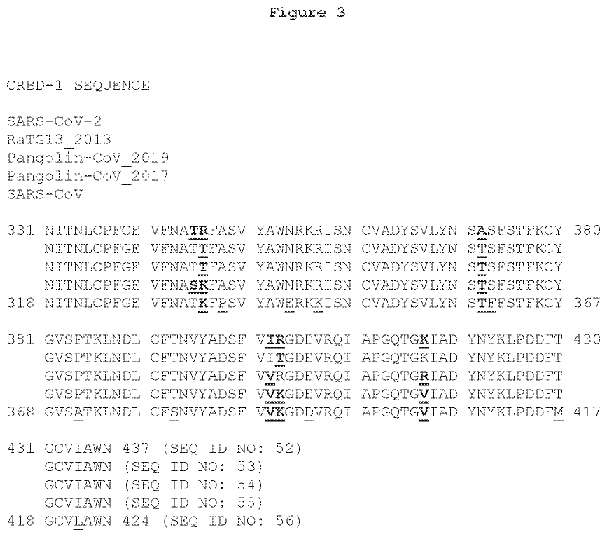

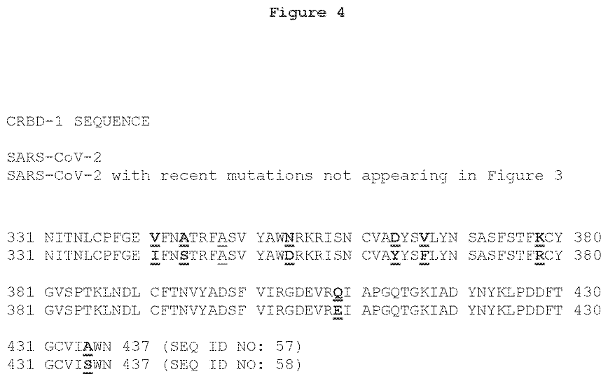

The conserved regions of the RBD (CRBD) are the regions flanking the receptor-binding motif (RBM) of the RBD (see FIG. 1) that makes complete contact with the receptor ACE2, with the addition of the conserved adjacent residue Asn424 in SARS-CoV or Asn437 in SARS-CoV-2, and the conserved adjacent GYQPY sequence of residues 490-494 in SARS-CoV or residues 504-508 in SARS-CoV-2. The CRBD consists of two fragments, one fragment CRBD-1 of 107 residues spanning residues 318-424 in SARS-CoV or residues 331-437 in SARS-CoV-2 and one fragment CRBD-2 of 21 residues spanning residues 490-510 in SARS-CoV or residues 504-524 in SARS-CoV-2 (see FIG. 2).

We define the variable receptor-binding motif (VRBM) as the RBM without the conserved residue Asn424 in SARS-CoV or Asn437 in SARS-CoV-2 and without the conserved GYQPY sequence of residues 490-494 in SARS-CoV or residues 504-508 in SARS-CoV-2. The VRBM spans residues 425-489 in SARS CoV spike protein and residues 438-503 in SARS CoV-2 spike protein.

As used herein, the RBD consists of 3 adjacent regions: CRBD-1, VRBM and CRBD-2.

Analysis of the CRBD-1 in betacoronaviruses infecting various species indicates that the CRBD-1 is also well conserved across betacoronaviruses infecting various species. This analysis allows the design of CRBD-1 variants for treating or limiting severe acute respiratory syndrome (SARS) coronavirus infection.

In another embodiment, the polypeptides of the disclosure include no more than 5, 4, 3, 2, or 1 residue from a SARS coronavirus spike protein VRBM, or include no amino acid residues from a SARS coronavirus spike protein VRBM. In one specific embodiment, the polypeptides of the disclosure do not include any portion of the VRBM of a SARS coronavirus spike protein, which contains 71 residues in contact with the receptor ACE2 in SARS-CoV, or 72 residues in contact with the receptor ACE2 in SARS-CoV-2. Thus, in another embodiment the polypeptides of the disclosure do not bind to the ACE2 receptor with any affinity. In a further embodiment, the polypeptides of the disclosure do not include a functional VRBM (meaning a VRBM able to bind to the ACE2 receptor with some affinity) from a SARS coronavirus spike protein. In a further specific embodiment, the CRBD of any embodiment disclosed herein is the only SARS coronavirus spike protein domain in the polypeptide. In a further embodiment, the polypeptide comprises a SARS coronavirus spike protein domain that consists of the CRBD of any embodiment disclosed herein.

The use of the CRBD-1 without the SARS spike protein receptor binding motif for treating or limiting SARS coronavirus infection is inventive, as those of skill in the art would be motivated to use most of the spike protein VRBM in order to compete with the binding to the ACE2 receptor in any vaccine or therapeutic for treating or limiting SARS coronavirus infection.

### Betacoronaviruses Infecting Bats and Pangolins are Likely Indirect Progenitors of SARS-CoV-2.

The bat Rhinoloffus affinis and the Malayan pangolin Manis javanica are infected by betacoronaviruses similar to SARS-CoV-2 and SARS-CoV. RaTG13, a bat coronavirus sampled from a Rhinolophus affinis in 2013, is 96% identical overall to SARS-CoV-2 for its protein amino acid sequences. Pangolin coronaviruses sampled in 2017 and 2019 exhibit particularly strong similarity to SARS-CoV-2 in the RBD, including all six key RBD residues that make contact with the receptor ACE2. This clearly shows that the SARS-CoV-2 spike protein optimized for binding to human-like ACE2 is the result of natural selection.

Neither the bat betacoronaviruses nor the pangolin betacoronaviruses sampled thus far can be a direct progenitor of SARS-CoV-2 because they lack some key features, such as polybasic cleavage sites for the pangolins and RBD residues in contact with ACE2 for the bats. Although no animal coronavirus has been identified that is sufficiently similar to have served as the direct progenitor of SARS-CoV-2, the diversity of coronaviruses in bats and other species is massively undersampled. Therefore natural selection in an animal host before zoonotic transfer is the likely origin of SARS-CoV-2.

### Comparison of CRBD-1 Across Species.

The CRBD-1 is well conserved across bat and pangolin betacoronaviruses that are close precursors of the human SARS-CoV-2 as indicated in FIG. 3. There are only 3 mutations in the bat RaTG13 virus observed in 2013 at positions R346T, A372T and R403T. There are only 4 mutations in the pangolin betacoronavirus observed in 2019 at positions R346T. A372T. I402V and K417R, and they are identical in the two samples. There are only 6 mutations in the pangolin betacoronavirus observed in in 2017 at positions T345S, R346K, A372T, I402V, R403K and K417V. and they are identical in all five samples. The CRBD-1 is a fragment of 107 residues therefore the mutation rate is respectively 3% for the bat RaTG13 virus, 4% for the 2019 pangolin betacoronavirus and 6% for the 2017 pangolin betacoronavirus.

A second aspect of CRBD-1 variation is that it is concentrated on 5 major residues of the CRBD-1 in positions Arg346, Ala372, Ile402, Arg403 and Lys417.

A third aspect of CRBD-1 variation is that most observed mutations are reverse mutations reverting to the original residue of SARS-CoV. Examples of such reverse mutations are as follows:
i) for the 2017 pangolin betacoronavirus: R346K, A372T, 1402V, R403K and K417V, or 5 out of 6 observed mutations;
ii) for the 2019 pangolin betacoronavirus: A372T and 1402V, or 2 out of 4 observed mutations;
iii) for the 2013 RaTG13 bat betacoronavirus: A372T, or 1 out of 3 observed mutations.

Two reverse mutations are particularly frequent. The I402V reverse mutation appears in all pangolins and the A372T reverse mutation appears in all bats and all pangolins.

A fourth aspect of the CRBD-1 variation is that the reverse mutation A372T reintroduces the glycosylation sequon NST that was lost in SARS-CoV-2 and therefore reintroduces the N-linked glycan at position 370 in SARS-CoV-2 that was lost at position 357 in SARS-CoV. Strikingly this reverse mutation appears in all the near precursors of SARS-CoV-2, including all sequences of pangolin betacoronaviruses observed in 2017 and 2019, the bat sequence of RaTG13 observed in 2013, and all observed sequences of bat betacoronaviruses in 2019 in two samples.

### Observed Mutations in Bats and Pangolins as Predictors of SARS-CoV-2 Evolution

The bat betacoronavirus RaTG13 observed in 2013 was a strong predictor of the SARS-CoV-2 as its protein amino acid sequences are 96% identical overall. However it is only 78% identical for the receptor-binding motif (RBM). The pangolin coronavirus as observed in 2017 and 2019 was a less accurate predictor of the SARS-CoV-2 with 91% identity of the S protein. However, it was an excellent predictor of the RBM with 98% identity, and 100% identity for the six key residues that make contact with the host receptor ACE2. The RaTG13 sequence as observed in 2013 combined with the pangolin betacoronavirus sequence as observed in 2017 allowed for a very accurate prediction of SARS-CoV-2, including for the highly variable RBM, at least two years before the outbreak of the SARS-CoV-2 pandemic.

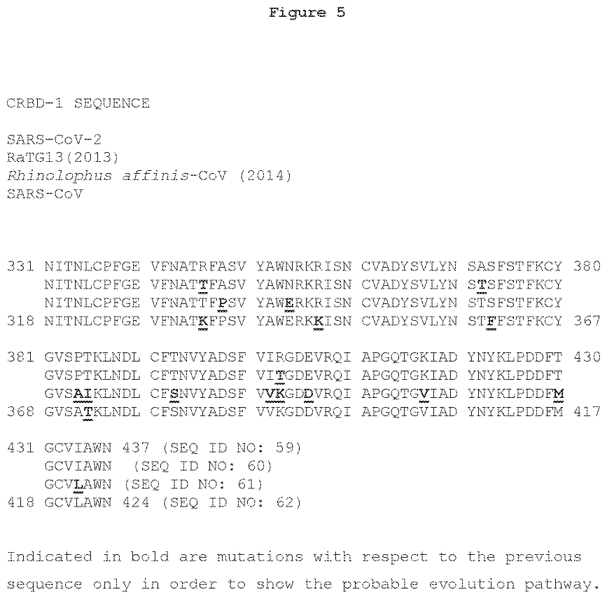

The evolution of the betacoronavirus in Rhinoloffus affinis can be further analyzed with a sequence observed in 2014 as indicated in FIG. 5. For the CRBD-1, as stated before, there are 3 mutations between the bat RaTG13 virus and SARS-CoV-2. For the CRBD-1 there are only 4 mutations between the betacoronavirus sequence observed in Rhinoloffus affinis in 2014 and SARS-CoV. There are 11 mutations between the sequences of RaTG13 observed in 2013 and the bat betacoronavirus observed in 2014. Therefore the betacoronaviruses observed in bats in 2013 and 2014 appear as intermediaries in the evolution of CRBD-1 between the two human SARS coronaviruses of 2003 and 2019. As expected the bat coronavirus of 2014 also harbors the reverse mutation A372T.

The recent evolution of the betacoronavirus in Rhinoloffus affinis and Manis javanica species indicates the current status of close precursors of SARS-CoV-2 before a potential new zoonotic transfer to humans. Applied to the CRBD-1 these observations can be summarized as follows:
i) R346K, A372T, I402V, R403K and K417V are frequent mutations in near precursors of SARS-CoV-2, reverting to SARS-CoV residues;
ii) A372T is a reverse mutation adding a third glycan on the CRBD-1 at position 370 with potential major change of immunogenicity of the CRBD-1 antigen. This mutation appears in all species and all observed samples of near precursors of SARS-CoV-2.

### Glycosylation of CRBD-1

The CRBD-1 polypeptide carries two N-linked glycans at position 331 (N-terminus) and 343. These two glycans have low oligomannose content below 30% and resemble the glycans in the host cells, and are believed to provide some camouflage from the host immune system. The addition of a third glycan on CRBD-1 at position 370 can therefore have a major effect on the immune response to the CRBD-1 antigen.

The design of a bivalent vaccine with one CRBD-1 polypeptide with a glycan at position 370 and one CRBD-1 polypeptide without this glycan can address the potential difference in immunogenicity of the CRBD-1 antigen with or without a glycan at position 370. The two CRBD-1 polypeptides of the bivalent vaccine may simply differ by the amino acid alanine or threonine at position 372.

Other mutations of CRBD-1

The following is a list of recently observed mutations in humans outside positions Arg346, Ala372, Ile402, Arg403 and Lys417: V341I, A344S, N354D, D364Y, V367F, K378R, Q409E and A435S.

FIG. 4 is providing the sequence of CRBD-1 with these mutations. These mutations are also incorporated into CRBD-1 variants.

Based on these analyses, the CRBDs of SEQ ID NOS:1-9 were designed. A summary of the CRBDs is provided below:

SEQ ID NO: 1: CRBD-1 with all mutations of SARS-CoV-2 recently observed in nature, in close progenitors and humans

SEQ ID NO:2: CRBD-1 with all mutations of SARS-CoV-2 observed in close progenitors and SARS-CoV

SEQ ID NO:3: CRBD-1 with all mutations of SARS-CoV-2, including close progenitors, humans, and SARS-CoV with A in position 372

SEQ ID NO:4: CRBD-1 with all mutations of SARS-CoV-2, including close progenitors, humans, and SARS-CoV with T in position 372

SEQ ID NO:5: Same as SEQ ID NO:2, with A in position 372

SEQ ID NO:6 Same as SEQ ID NO:2, with T in position 372

SEQ ID NO:7: CRBD-1 with all mutations between SARS-CoV and SARS-CoV-2

SEQ ID NO:8: CRBD-1 with all mutations of SARS-CoV-2, including recently observed in close progenitors and humans, and between SARS-CoV-2 and SARS-CoV

SEQ ID NO:9: CRBD-2

SEQ ID NO:38: CRBD-1 with all mutations of SARS-CoV-2 recently observed in nature, in close progenitors and humans and variants

In one embodiment, the CRBD comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-8 and 38. This embodiment limits the polypeptide to those comprising CRBD1. In another embodiment, the CRBD comprises an amino acid sequence at least 70%, 75%, 80%, 85%, 90%, 91%, 92%, 93%, 94%, 95%, 96%, 97%, 98/a, 99%, or 100% identical to the amino acid sequence of SEQ ID NO:10 or 22.

(SEQ ID NO: 10)
NITNLCPFGEVFNATRFASVYAWNRKRISNCVADYS
VLYNSASFSTFKCYGVSPTKLNDLCFTNVYADSFVI
RGDEVRQIAPGQTGKIADYNYKLPDDFTGCVIAWN
(SARS-CoV-2 CRBD-1)
(SEQ ID NO: 22)
NITNLCPFGEVFNATRFASVYAWNRKRISNCVADYS
VLYNSTSFSTFKCYGVSPTKLNDLCFTNVYADSFVI
RGDEVRQIAPGQTGKIADYNYKLPDDFTGCVIAWN
(SARS-CoV-2
CRBD-1 with mutation T in position 372;

In a further embodiment, differences in the CRBD amino acid sequence and SEQ ID NO:10 comprise differences at 1 or more positions selected from residues 11, 14, 15, 16, 18, 24, 27, 34, 37, 42, 43, 48, 54, 63, 72, 73, 76, 79, 87, 100, 104, and 105 in SEQ ID NO:10 or 22. These residues are shown by the above analyses to be variable between different CRBD-1 variants.

In another embodiment, the CRBD comprises:

(a) the amino acid sequence of SEQ ID NO:1, wherein at least 1, 2, 3, 4, 5, 6, 7, 8, 9, 10, 11, 12, 13, or all 14 of the residues bounded by parentheses is the first listed residue:

(SEQ ID NO: 1)
NITNLCPFGE(V/I)FN(A/S)(T/S)(R/T/K)FA
SVYAW(N/D)RKRISNCVA(D/Y)YS(V/F)LYNS(
A/T)SFSTF(K/R)CYGVSPTKLNDLCFTNVYADSF
V(I/V)(R/T/K)GDEVR(Q/E)IAPGQTG(K/R/V
)IADYNYKLPDDFTGCVI(A/S)WN;

(b) the amino acid sequence of SEQ ID NO:2, wherein at least 1, 2, 3, 4, 5, or all 6 of the residues bounded by parentheses is the first listed residue:

(SEQ ID NO: 2)
NITNLCPFGEVFNA(T/S)(R/T/K)FASVYAWNRK
RISNCVADYSVLYNS(A/T)SFSTFKCYGVSPTKLN
DLCFTNVYADSFV(I/V)(R/T/K)GDEVRQIAPGQ
TG(K/R/V)IADYNYKLPDDFTGCVIAWN;

(c) the amino acid sequence of SEQ ID NO:3 or SEQ ID NO: wherein 1, 2, 3, 4, 5, 6, 7, 8, 9, 10, 11, 12, 13, 14, 15, 16, 17, 18, 19, 20 or all 21 of the residues bounded by parentheses is the first listed residue:

(SEQ ID NO: 3)
NITNLCPFGE(V/I)FN(A/S)(T/S)(R/T/K)F(
A/P)SVYAW(N/E/D)RK(R/K)ISNCVA(D/Y)YS
(V/F)LYNSA(S/F)FSTF(K/R)CYGVS(P/A)TK
LNDLCF(T/S)NVYADSFV(I/V)(R/T/K)GD(E/
D)VR(Q/E)IAPGQTG(K/R/V)IADYNYKLPDDF(
T/M)GCV(I/L)(A/S)WN;
(SEQ ID NO: 4)
NITNLCPFGE(V/I)FN(A/S)(T/S)(R/T/K)F(
A/P)SVYAW(N/E/D)RK(R/K)ISNCVA(D/Y)YS
(V/F)LYNST(S/F)FSTF(K/R)CYGVS(P/A)TK
LNDLCF(T/S)NVYADSFV(I/V)(R/T/K)GD(E/
D)VR(Q/E)IAPGQTG(K/R/V)IADYNYKLPDDF(
T/M)GCV(I/L)(A/S)WN;

(d) the amino acid sequence of SEQ ID NO:5 or SEQ ID NO:6, wherein 1, 2, 3, 4, 5, or all 6 of the residues bounded by parentheses is the first listed residue:

(SEQ ID NO: 5)
NITNLCPFGEVFNA(T/S)(R/T/K)FASVYAWNRK
RISNCVADYSVLYNSASFSTFKCYGVSPTKLNDLCF
TNVYADSFV(I/V)(R/T/K)GDEVRQIAPGQTG(K
/R/V)IADYNYKLPDDFTGCVIAWN;
(SEQ ID NO: 6)
NITNLCPFGEVFNA(T/S)(R/T/K)FASVYAWNRK
RISNCVADYSVLYNSTSFSTFKCYGVSPTKLNDLCF
TNVYADSFV(I/V)(R/T/K)GDEVRQIAPGQTG(K
/R/V)IADYNYKLPDDFTGCVIAWN;

(e) the amino acid sequence of SEQ ID NO:7, wherein at least 1, 2, 3, 4, 5, 6, 7, 8, 9, 10, 11, 12, 13 or all 14 of the amino acid residues in SEQ ID NO:7, bounded by parentheses are the first listed residue

(SEQ ID NO: 7)
NITNLCPFGEVFNAT(R/K)F(A/P)SVYAW(N/E)
RK(R/K)TSNCVADYSVLYNS(A/T)(S/F)FSTFK
CYGVS(P/A)TKLNDLCF(T/S)NVYADSFV(I/V)
(R/K)GD(E/D)VRQIAPGQTG(K/V)IADYNYKLP
DDF(T/M)GCV(I/L)AWN;

or

(f) the amino acid sequence of SEQ ID NO: 8, wherein at least 1, 2, 3, 4, 5, 6, 7, 8, 9,10, 11, 12, 13, 14, 15, 16, 17, 18, 19, 20, 21, or all 22 of the residues bounded by parentheses is the first listed residue.

(SEQ ID NO: 8)
NITNLCPFGE(V/I)FN(A/S)(T/S)(R/T/K)F(
A/P)SVYAW(N/E/D)RK(R/K)ISNCVA(D/Y)YS
(V/F)LYNS(A/T)(S/F)FSTF(K/R)CYGVS(P/
A)TKLNDLCF(T/S)NVYZDSFV(I/V)(R/T/K)G
D(E/D)VR(Q/E)IAPGQTG(K/R/V)IADYNYKLP
DDF(T/M)GCV(I/I)(A/S)WN

In a specific embodiment, the CRBD comprises the amino acid sequence of SEQ ID NO:2, wherein at least 1, 2, 3, 4, 5, or all 6 of the amino acid residues in SEQ ID NO:2 bounded by parentheses are the first listed residue. In another specific embodiment, the CRBD comprises the amino acid sequence of SEQ ID NO: 2, SEQ ID NO: 10, or SEQ ID NO:22.

In another embodiment, the CRBD comprises an amino acid sequence at least 70%, 75%, 80%, 85%, 90%, 91%, 92%, 93%, 94%, 95%, 96%, 97%, 98%, 99%, or 100% identical to SEQ ID NO:9. In this embodiment, the CRBD comprises CRBD-1. In specific embodiments, the CRBD comprises the amino acid sequence of SEQ ID NO:9 or SEQ ID NO:25.

### GYQPYRVVVLSFELLHAPATV (SEQ ID NO:25) (SARS-CoV-2 CRBD-2)

The polypeptide may comprise one or more copies of the CRBD. In one embodiment, the CRBD is present in 2, 3, 4, 5, 6, 7, 8, 9, 10 or more copies.

In another embodiment, the polypeptides of the disclosure further comprise a multimerization domain. The polypeptides can be engineered via genetic fusion to create 3-mer, 4-mer and 8-mer multimers. These constructs may be expressed, for example, in Chinese hamster ovary (CHO) cells and purified using standard nickel and size exclusion methods. By size exclusion chromatography with multi-angle light scattering (SEC-MALS), each construct is shown to have the correct molecular weight according to its intended multimeric state. The antigenic profiles of the constructs are tested and the results show binding to neutralizing antibodies.

In this embodiment, the polypeptide is capable of multimerization and thus presenting multiple copies of the CRBD to enhance the immune response generated when the polypeptide is administered to a subject. Any multimerization can be used as is deemed suitable for an intended use. In one embodiment, the multimerization domain comprises an amino acid sequence at least at least 70%, 75%, 80%, 85%, 90%, 91%, 92%, 93%, 94%, 95%, 96%, 97%, 98%, 99%, or 100% identical to the amino acid sequence of SEQ ID NO: 23, 24, 27, or 28.

(SEQ ID NO: 23)
MQIY(E/C)GK(L/C)(T/G)AEGLRFGIVASR(F/
A)NHALVDRLVEGAIDAIV(R/C)(H/F/M)GGREE
DITLV(R/C)V(P/C)GSWEIP(V/C)AAGELARKE
DIDAVIAIGVL(I/C)RGA(T/C)(P/G)(H/S)FD
YIASEVSKGLADLS(L/C)ELRKPITFGVITA(D/C
)TLBQAIE(R/A)AGT(K/C)HGNKGWEAAL(S/C)
AIEMANLFKSLR
(Lumazine synthase (LS) variants)
(SEQ ID NO: 24)
MQIYEGKLTAEGLRFGIVASRANHALVDRLVEGAID
AIVRHGGREEDITLVRVCGSWEIPVAAGELARKEDI
DAVIAIGVLCRGATPSFDYIASEVSKGLADLSLELR
KPITFGVITADTLEQAIEAAGTCHGNKGWEAALCAI
EMANLFKSLR
(Lumazine synthase (LS))

In this embodiment, the multimerization platform comprises lumazine synthase. The multimerization domains of SEQ ID NOS: 23 and 24 can be used to generate multimers comprising 60 copies of the isolated polypeptides of the disclosure.

(SEQ ID NO: 27)
RMKQIEDKIEEILSKIYHIENEIARIKKLIGER
(Coiled trimerization motif)
(SEQ ID NO: 28)
MKVKQLEDVVEELLSVNYHLENVVARLKKLVGER
(Tetramerization motif having 4 helices curling
around each other in helical manner)

The multimerization domains of SEQ ID NOS: 27 and 28 can be used to generate multimers comprising 3, 4, 6 or 8 copies of the isolated polypeptides of the disclosure.

In one embodiment where the linker comprises SEQ ID NO:23, at least 1, 2, 3, 4, 5, 6, 7, 8, 9,10, 11, 12, 13, 14, 15 or all 16 of the residues bounded by parentheses is the first listed residue.

In a further embodiment, the polypeptide comprises a SARS coronavirus spike protein domain that consists of the CRBD of any embodiment disclosed herein, and a multimerization domain of any embodiment disclosed herein.

In another embodiment the polypeptides of the disclosure may further comprise an amino acid linker between the CRBD domain and the multimerization domain. Any amino acid linker may be used as suitable for an intended purpose. In one embodiment, the linker is a Gly-Ser rich linker (i.e.: 50%, 60%, 70%, 80%, 90%, 95%, or 100% made up of Gly or Ser residues). The combination of flexible and hydrophilic residues in these linkers limits the formation of secondary structures and reduces the likelihood that the linkers will interfere with the folding and function of the protein domains. In one specific embodiment, the linker comprises or consists of (GGS)ₙGGG, wherein n is 3-7, 3-6, 3-5, 3-4, 4-7, 4-6, 4-5, 5-7, 5-6, 3, 4, 5, 6, or 7.

The multimerization may be N-terminal or C-terminal to the CRBD. In one specific embodiment, the CRBD is carboxy-terminal to the multimerization domain.

In other embodiments, the polypeptide comprises the amino acid sequence selected from the group consisting of SEQ ID NOS: 11-18 and 39, wherein n is 3-7, 3-6, 3-5, 3-4, 4-7, 4-6, 4-5, 5-7, 5-6, 3, 4, 5, 6, or 7.

(SEQ ID NO: 11)
MQIY (E/C) GK (L/C) (T/G) AEGLRFGIVASR(F/A)
NHALVDRLVEGAIDAIV (R/C) (H/F/M) GGREEDITLV
(R/C) V (P/C) GSWEIP (V/C) AAGELARKEDIDAVIAIGVL
(I/C) RGA (T/C)(P/G) (H/S) FDYIASEVSKGLADLS (L/C)
ELRKPITFGVITA (D/C) TLEQAIE (R/A) AGT (K/C) HGN
KGWEAAL (S/C) AIEMANLFKSLR (GGS) ₙGGGNITNLCPFGE
(V/I) FN (A/S ) (T/S) (R/T/K) FASVYAW (N/D)
RKRISNCVA (D/Y) YS (V/F) LYNS (A/T) SFSTF (K/R)
CYGVSPTKLNDLCFTNVYADSFV (I/V) (R/T/K) GDEVR
(Q/E) IAPGQTG (K/R/V) IADYNYKLPDDFTGCVI (A/S) WN
CRBD-1 including all mutations of SARS-CoV-2
recently observed in nature, in close progenitors
and humans
(SEQ ID NO: 12)
MQIY (E/C) GK (L/C) (T/G) AEGLRFGIVASR (F/A)
NHALVDRLVEGAIDAIV (R/C) (H/F/M) GGREEDITLV
(R/C) V (P/C) GSWEIP (V/C AAGELARKEDIDAVIAIGVL
(I/C) RGA (T/C) (P/G) (H/S) FDYIASEVSKGLADLS (L/C)
ELRKPITFGVITA (D/C) TLEQAIE (R/A) AGT (K/C) HGN
KGWEAAL (S/C) AIEMANLFKSLR (GGS) ₙGGGNITNLCPFGEVF
NA (T/S) (R/T/K) FASVYAWNRKRISNCVADYSVLYNS (A/T)
SFSTFKCYGVSPTKLNDLCFTNVYADSFV (I/V) (R/T/K) GDEVR
QIAPGQTG (K/R/V) IADYNYKLPDDFTGCVIAWN
CRBD-1 including mutations of SARS-CoV-2
observed in close progenitors and with SARS-CoV
(SEQ ID NO: 13)
MQIY (E/C) GK(L/C) (T/G) AEGLRFGIVASR (F/A)
NHALVDRLVEGAIDAIV (R/C) (H/F/M) GGREEDITLV
(R/C) V(P/C) GSWEIP (V/C) AAGELARKEDIDAVIAIGVL
(I/C) RGA (T/C) (P/G) (H/S) FDYIASEVSKGLADLS (L/C)
ELRKPITFGVITA (D/C) TLEQAIE (R/A) AGT (K/C) HGN
KGWEAAL (S/C) AIEMANLFKSLR (GGS) ₙGGGNITNLCPFGE
(V/I) FN (A/S) (T/S) (R/T/K) F (A/P) SVYAW (N/E/D)
RK (R/K) ISNCVA (D/Y) YS (V/F) LYNSA (S/F) FSTF
(K/R) CYGVS (P/A) TKLNDLCF (T/S) NVYADSFV (I/V)
(R/T/K) GD (E/D) VR (Q/E) IAPGQTG (K/R/V) IADY
NYKLPDDF (T/M) GCV (I/L) (A/S) WN
CRBD-1 including all mutations of SARS-CoV-2,
including in close progenitors, humans, and
SARS-CoV with A in position 372
(SEQ ID NO: 14)
MQIY (E/C) GK (L/C) (T/G) AEGLRFGIVASR (F/A)
NHALVDRLVEGAIDAIV (R/C) (H/F/M) GGREEDITLV
(R/C) V (P/C) GSWEIP (V/C) AAGELARKEDIDAVIAIGVL
(I/C) RGA (T/C) (P/G) (H/S) FDYIASEVSKGLADLS (L/C)
ELRKPITFGVITA (D/C) TLEQAIE (R/A) AGT (K/C) HGN
KGWEAAL (S/C) AIEMANLFKSLR (GGS) ₙGGGNITNLCPFGE
(V/I) FN (A/S) (T/S) (R/T/K) F (A/P) SVYAW (N/E/D)
RK (R/K) ISNCVA (D/Y) YS (V/F) LYNST (S/F) FSTF
(K/R) CYGVS (P/A) TKLNDLCF (T/S) NVYADSFV (I/V)
(R/T/K) GD (E/D) VR (Q/E) IAPGQTG (K/R/V) IADY
NYKLPDDF (T/M) GCV (I/L) (A/S) WN
CRBD-1 including all mutations of SARS-CoV-2,
including in close progenitors, humans, and
SARS-CoV with T in position 372.
(SEQ ID NO: 15)
MQIY (E/C) GK (L/C) (T/G) AEGLRFGIVASR (F/A)
NHALVDRLVEGAIDAIV (R/C) (H/F/M) GGREEDITLV
(R/C) V (P/C) GSWEIP (V/C) AAGELARKEDIDAVIAIGVL
(I/C) RGA (T/C) (P/G) (H/S) FDYIASEVSKGLADLS (L/C)
ELRKPITFGVITA (D/C) TLEQAIE (R/A) AGT (K/C) HGN
KGWEAAL (S/C) AIEMANLFKSLR (GGS) ₙGGGNITNLCPFGEVF
NA (T/S) (R/T/K) FASVYAWNRKRISNCVADYSVLYNSA
SFSTFKCYGVSPTKLNDLCFTNVYADSFV (I/V) (R/T/K)
GDEVRQIAPGQTG (K/R/V) IADYNYKLPDDFTGCVIAWN
SEQ ID NO: 12 with A in position 372
(SEQ ID NO: 16)
MQIY (E/C) GK (L/C) (T/G) AEGLRFGIVASR (F/A)
NHALVDRLVEGAIDAIV (R/C) (H/F/M) GGREEDITLV
(R/C) V (P/C) GSWEIP (V/C) AAGELARKEDIDAVIAIGVL
(I/C) RGA (T/C) (P/G) (H/S) FDYIASEVSKGLADLS (L/C)
ELRKPITFGVITA (D/C) TLEQAIE(R/A) AGT (K/C) HGNKG
WEAAL (S/C) AIEMANLFKSLR (GGS) ₙGGGNITNLCPFGEVFNA
(T/S) (R/T/K) FASVYAWNRKRISNCVADYSVLYNSTS
FSTFKCYGVSPTKLNDLCFTNVYADSFV (I/V) (R/T/K)
GDEVRQIAPGQTG (K/R/V) IADYNYKLPDDFTGCVIAWN
SEQ ID NO: 12 with T in position 372
LS-GS-CRBD-1
(SEQ ID NO: 17)
MQIY (E/C) GK (L/C) (T/G) AEGLRFGIVASR (F/A)
NHALVDRLVEGAIDAIV (R/C) (H/F/M) GGREEDITLV
(R/C) V (P/C) GSWEIP (V/C) AAGELARKEDIDAVIAIGVL
(I/C) RGA (T/C) (P/G) (H/S) FDYIASEVSKGLADLS
(L/C) ELRKPITFGVITA (D/C) TLEQAIE (R/A) AGT (K/C)
HGNKGWEAAL (S/C) AIEMANLFKSLR (GGS) ₙGGGNITNLCPF
GEVFNAT (R/K) F (A/P) SVYAW (N/E) RK (R/K)
ISNCVADYSVLYNS (A/T) (S/F) FSTFKCYGVS (P/A)
TKLNDLCF (T/S) NVYADSFV (I/V) (R/K) GD (E/D)
VRQIAPGQTG (K/V) IADYNYKLPDDF (T/M) GCV (I/L) AWN
CRBD-1 including all mutations between SARS-CoV
and SARS-CoV-2
LS-GS-CRBD-2
(SEQ ID NO: 18)
MQIY (E/C) GK (L/C) (T/G) AEGLRFGIVASR (F/A)
NHALVDRLVEGAIDAIV (R/C) (H/F/M) GGREEDITLV
(R/C) V (P/C) GSWEIP (V/C) AAGELARKEDIDAVIAIGVL
(I/C) RGA (T/C) (P/G) (H/S) FDYIASEVSKGLADLS (L/C)
ELRKPITFGVITA (D/C) TLEQAIE (R/A) AGT (K/C) HGN
KGWEAAL (S/C) AIEMANLFKSLR (GGS) ₙGGGGYQPYRVVVLSF
ELL (H/N) APATV.
CRBD-2 including all mutations between SARS-CoV
and SARS-Cov-2
(SEQ ID NO: 19)
MQIY (E/C) GK (L/C) (T/G) AEGLRFGIVASR (F/A)
NHALVDRLVEGAIDAIV (R/C) (H/F/M) GGREEDITLV
(R/C) V (P/C) GSWEIP (V/C) AAGELARKEDIDAVIAIGVL
(I/C )RGA (T/C) (P/G) (H/S) FDYIASEVSKGLADLS (L/C)
ELRKPITFGVITA (D/C) TLEQAIE (R/A) AGT (K/C) HGN
KGWEAAL (S/C) AIEMANLFKSLR (GGS) ₙGGGNITNLCPFGE
(V/I) FN (A/S) (T/S) (R/T/K) F (A/P) SVYAW (N/E/
D) RK (R/K) ISNCVA (D/Y) YS (V/F) LYNS (A/T) (S/F)
FSTF (K/R) CYGVS (P/A) TKLNDLCF (T/S) NVYADSFV
(I/V) (R/T/K) GD (E/D) VR (Q/E) IAPGQTG (K/R/V)
IADYNYKLPDDF (T/M) GCV (I/L) (A/S) WN
CRBD-1 including all imitations of SARS-CoV-2,
including recently observed in close progenitors
and humans, and between SARS-CoV-2 and SARS-CoV
(SEQ ID NO: 39)
MQIY (E/C) GK (L/C) (T/G) AEGLRFGIVASR (F/A)
NHALVDRLVEGAIDAIV (R/C) (H/F/M) GGREEDITLV
(R/C) V (P/C) GSWEIP (V/C) AAGELARKEDIDAVIAIGVL
(I/C) RGA (T/C)(P/G) (H/S) FDYIASEVSKGLADLS (L/C)
ELRKPITFGVITA (D/C) TLEQAIE (R/A) AGT (K/C)HGN
KGWEAAL (S/C) AIEMANLFKSLR (GGS) ₙGGGNITNLCPFGE
(V/I) FN (A/S) (T/S) (R/T/K) F ASVYAW (N/D)
RKRISNCVA (D/Y) YS (V/F) LYNS (A/T) SFSTF (K/R)
CYGVSPTKLNDL (C/A) FTNVYADSFV (I/V) (R/T/K) GDEVR
(Q/E) IAPGQTG (K/R/V/N/T) IADYNYKLPDDFTGCVI
(A/S)WN
CRBD-1 including all mutations of SARS-CoV-2
recently observed in nature, in close
progenitors and human, and variants

In specific embodiments, the polypeptide comprises the amino acid sequence of SEQ ID NO:20, 21, or 22.

(SEQ ID NO: 20)
MQIYEGKLTAEGLRFGIVASRANHALVDRLVEGAIDAIVRHGGRE
EDITLVRVCGSWEIPVAAGELARKEDIDAVIAIGVLCRGATPSFD
YIASEVSKGLADLSLELRKPITFGVITADTLEQAIEAAGTCHGNK
GWEAALCAIEMANLFKSLRGGSGGSGGSGGSGGGNITNLCPFGEV
FNATRFASVYAWNRKRISNCVADYSVLYNSASFSTFKCYGVSPTK
LNDLCFTNVYADSFVIRGDEVRQIAPGQTGKIADYNYKLPDDFTG
CVIAWN
SARS-CoV-2 sequence
(SEQ ID NO: 21)
MQIYEGKLTAEGLRFGIVASRANHALVDRLVEGAIDAIVRHGGRE
EDITLVRVCGSWEIPVAAGELARKEDIDAVIAIGVLCRGATPSFD
YIASEVSKGLADLSLELRKPITFGVITADTLEQAIEAAGTCHGNK
GWEAALCAIEMANLFKSLRGGSGGSGGSGGSGGGNITNLCPFGEV
FNATRFASVYAWNRKRISNCVADYSVLYNSTSFSTFKCYGVSPTK
LNDLCFTNVYADSFVIRGDEVRQIAPGQTGKIADYNYKLPDDFTG
CVIAWN
SARS-CoV-2 sequence with mutation T in
position 372
(SEQ ID NO: 32)
MQIYEGKLTAEGLRFGIVASRANHALVDRLVEGAIDAIVRHGGRE
EDITLVRVCGSWEIPVAAGELARKEDIDAVIAIGVLCRGATPSFD
YIASEVSKGLADLSLELRKPITFGVITADTLEQAIEAAGTCHGNK
GWEAALCAIEMANLFKSLRGGSGGSGGSGGSGGGNITNLCPFGEV
FNATRFASVYAWNRKRISNCVADYSVLYNSASFSTFKCYGVSPTK
LNDLAFTNVYADSFVIRGDEVRQIAPGQTGKIADYNYKLPDDFTG
CVIAWN
SARS-CoV-2 sequence with mutation A in
position 230

In another embodiment, the isolated polypeptide comprises the general formula selected from

X1-(GGS)ₙGGG-X3 and X 1-(GGS)ₙGGG-X3-(GGS)ₙGGG-X2, wherein

X1 and X2 independently comprise the amino acid sequence selected from the group consisting SEQ ID NOS:1-10, 22, and 25;

n is 3-5, 3-4, 4-5, 3, 4, 5 and

X3 comprises the amino acid sequence selected from the group consisting SEQ ID NO:27 or 28.

In embodiments where the general formula is X1-(GGS)ₙGGG-X3, the polypeptide is capable of trimerization (when X3 comprises SEQ ID NO:27) or tetramerization (when X3 comprises SEQ ID NO:28).

In embodiments where the general formula is X1-(GGS)ₙGGG-X3-(GGS)ₙGGG-X2, the polypeptide is capable of forming multimers including an octamer when X3 comprises SEQ ID NO:28.

In all of these embodiments, X1 and X2 may comprise any one of SEQ ID NOS:1-10, 22, and 25. In one embodiment, where the general formula is X1-(GGS)ₙGGG-X3-(GGS)ₙGGG-X2, X1 and X2 comprise the same CRBD. In other embodiments where the general formula is X1-(GGS)ₙGGG-X3-(GGS)ₙGGG-X2, X1 and X2 comprise different CRBDs, providing a bivalent construct.

In one embodiment, X1 and X2 (when present) independently comprise an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NOS:1-8 and 38. This embodiment limits the polypeptide to those comprising CRBD1. In another embodiment, X1 and X2 (when present) independently an amino acid sequence at least 70%, 75%, 80%, 85%, 90%, 91%, 92%, 93%, 94%, 95%, 96%, 97%, 98%, 99%, or 100% identical to the amino acid sequence of SEQ ID NO:10 or 22, optionally wherein the X2 and X2 amino acid sequence and SEQ ID NO:10 comprise differences at 1 or more positions selected from residues 11, 14, 15, 16, 18, 24, 27, 34, 37, 42, 43, 48, 54, 63, 72, 73, 76, 79, 87, 100, 104, and 105 in SEQ ID NO:10 or 22. In a further embodiment, X1 and X2 (when present) independently comprise:

(a) the amino acid sequence of SEQ ID NO: 1, wherein at least 1, 2, 3, 4, 5, 6, 7, 8, 9, 10, 11, 12, 13, or all 14 of the residues bounded by parentheses is the first listed residue:

(b) the amino acid sequence of SEQ ID NO:2, wherein at least 1, 2, 3, 4, 5, or all 6 of the residues bounded by parentheses is the first listed residue;

(c) the amino acid sequence of SEQ ID NO:3 or SEQ ID NO:4, wherein 1, 2, 3, 4, 5, 6, 7, 8, 9,10, 11, 12, 13, 14, 15, 16, 17, 18, 19, 20 or all 21 of the residues bounded by parentheses is the first listed residue;

(d) the amino acid sequence of SEQ ID NO:5 or SEQ ID NO:6, wherein 1, 2, 3, 4, 5, or all 6 of the residues bounded by parentheses is the first listed residue;

(e) the amino acid sequence of SEQ ID NO:7, wherein at least 1, 2, 3, 4, 5, 6, 7, 8, 9, 10, 11, 12, 13 or all 14 of the amino acid residues in SEQ ID NO:7 bounded by parentheses are the first listed residue; or

(f) the amino acid sequence of SEQ ID NO:8, wherein at least 1, 2, 3, 4, 5, 6, 7, 8, 9, 10, 11, 12, 13, 14, 15, 16, 17, 18, 19, 20, 21, or all 22 of the residues bounded by parentheses is the first listed residue.

In a specific embodiment, X1 and X2 (when present) independently comprise the amino acid sequence of SEQ ID NO:2, wherein at least 1, 2, 3, 4, 5, or all 6 of the amino acid residues in SEQ ID NO:2 bounded by parentheses are the first listed residue, or independently comprise the amino acid sequence of SEQ ID NO:2, SEQ ID NO:10, or SEQ ID NO:22.

In further specific embodiments, the polypeptide:

(a) comprises the amino acid sequence of SEQ ID NO:29, wherein 1, 2, 3, 4, 5, 6, 7, 8, 9,10, 11, 12, 13, 14, 15, 16, 17, 18, 19, 20, 21 or all 22 of the residues bounded by parentheses is the first listed residue:

(SEQ ID NO: 29)
NITNLCPFGEVFNA (T/S) (R/T/K) FASVYAWNRKRIS
NCVADYSVLYNS (A/T) SFSTFKCYGVSPTKLNDLCFTNV
YADSFV (I/V) (R/T/K) GDEVRQIAPGQTG (K/R/V)
IADYNYKLPDDPTGCVIAWNGGSGGSGGSGGGMKVKQLEDVV
EELLSVNYHLENVVARLKKLVGERGGSGGSGGSGGGNITNLC
PFGEVFNA (T/S) (R/T/K) FASVYAWNRKRISNCVADY
SVLYNS (A/T) SFSTFKCYGVSPTKLNDLCFTNVYADSFV
(I/V) (R/T/K) GDEVRQIAPGQTG (K/R/V)
IADYNYKLPDDFTGCVIAWN

(b) comprises the amino acid sequence of SEQ ID NO:30, wherein 1, 2, 3, 4, 5, 6, 7, 8, 9,10, 11, 12, 13, 14, 15, 16, 17, 18, 19, 20 or all 21 of the residues bounded by parentheses is the first listed residue:

(SEQ ID NO: 30)
NITNLCPFGEVFNA (T/S) (R/T/K) FASVYAWNRKRIS
NCVADYSVLYNSASFSTFKCYGVSPTKLNDLCFTNVYADSFV
(I/V) (R/T/K) GDEVRQIAPGQTG (K/R/V)
IADYNYKLPDDFTGCVIAWNGGSGGSGGSGGGMKVKQLEDVV
EELLSVNYHLENVVARLKKLVGERGGSGGSGGSGGGNITNLC
PFGEVFNA (T/S) (R/T/K) FASVYAWNRKRISNCVADY
SVLYNSTSFSTFKCYGVSPTKLNDLCFTNVYADSFV
(I/V) (R/T/K) GDEVRQIAPGQTG (K/R/V)
IADYNYKLPDDFTGCVIAWN

(c) comprises the amino acid sequence of SEQ ID NO:31, wherein 1, 2, 3, 4, 5, 6, 7, 8, 9,10, 11, 12, 13, 14, 15, 16, 17, 18, 19, 20 or all 21 of the residues bounded by parentheses is the first listed residue:

(SEQ ID NO: 31)
NITNLCPFGEVFNA (T/S) (R/T/K) FASVYAWNRKRI
SNCVADYSVLYNSTSFSTFKCYGVSPTKLNDLCFTNVYADSFV
(I/V) (R/T/K) GDEVRQIAPGQTG (K/R/V)
IADYNYKLPDDFTGCVIAWNGGSGGSGGSGGGMKVKQLEDVVE
ELLSVNYHLENVVARLKKLVGERGGSGGSGGSGGGNITNLCPF
GEVFNA (T/S) (R/T/K) FASVYAWNRKRISNCVADYSVL
YNSASFSTFKCYGVSPTKLNDLCFTNVYADSFV (I/V)
(R/T/K) GDEVRQIAPGQTG (K/R/V) IADYNYKLPDDF
TGCVIAWN

In another aspect, the disclosure provides multimers, comprising two or more copies of the isolated polypeptide of any embodiment or combination of embodiments disclosed herein. The multimers may be formed in any suitable manner, including but not limited to by inclusion of multimerization domains in the primary amino acid sequence, or by linking the polypeptides to a scaffold. In one embodiment, the multimer comprises between 2 and 60 copies of the isolated polypeptide. In various embodiments, the multimer may comprise 2, 3, 4, 6, 8, 60, or more copies of the polypeptide. In one embodiment, the disclosure provides scaffolds comprising two or more isolated polypeptides of any embodiment or combination of embodiments disclosed herein on a surface of the scaffold. Any suitable scaffolds may be used, whether polypeptide scaffolds, virus-like particles, beads, or other scaffold materials. The polypeptides may be linked to the scaffolds in any suitable matter. In one embodiment, the two or more isolated polypeptides are all identical polypeptides. In another embodiment, the two or more isolated polypeptides include different polypeptides, permitting delivery of a multivalent composition to a subject in need thereof. In non-limiting and exemplary such embodiments, the different polypeptides present on a scaffold comprise:

(a) the CRBD of SEQ ID NO:3 and the CRBD of SEQ ID NO:4;

(b) the isolated polypeptide of SEQ ID NO:13 and the isolated polypeptide of SEQ ID NO:14:

(c) the CRBD of SEQ ID NO:5 and the CRBD of SEQ ID NO:6;

(d) the isolated polypeptide of SEQ ID NO:15 and the isolated polypeptide of SEQ ID NO:16; and/or

(e) two or all three of the isolated polypeptide of SEQ ID NO:20, the isolated polypeptide of SEQ ID NO:21, and the isolated polypeptide of SEQ ID NO:32.

In another aspect, the disclosure provides isolated nucleic acids encoding the isolated polypeptide of any embodiment or combination of embodiments disclosed herein. The isolated nucleic acid sequence may comprise RNA or DNA. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In another aspect, the present disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors include but are not limited to, plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector (including but not limited to a retroviral vector or oncolytic virus), or any other suitable expression vector. In some embodiments, the expression vector can be administered in the methods of the disclosure to express the polypeptides in vivo for therapeutic benefit.

In a further aspect, the present disclosure provides host cells that comprise the polypeptides, nucleic acids, expression vectors and/or nucleic acids disclosed herein, wherein the host cells can be either prokaryotic or eukaryotic. The cells can be transiently or stably engineered to incorporate the expression vector of the invention, using techniques including but not limited to bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press); Culture of Animal Cells: A Manual of Basic Technique, 2^{nd} Ed. (R. I. Freshney, 1987. Liss, Inc. New York, N.Y.)). A method of producing a polypeptide according to the invention is an additional part of the invention. The method comprises the steps of (a) culturing a host according to this aspect of the invention under conditions conducive to the expression of the polypeptide, and (b) optionally, recovering the expressed polypeptide. The expressed polypeptide can be recovered from the cell free extract, but preferably they are recovered from the culture medium.

In one embodiment of the nucleic acids of the disclosure, the nucleic acid comprises mRNA. Messenger RNA (mRNA) offers a relatively safe and efficient alternative to the polypeptide therapeutics and vaccines of the disclosure. After mRNA in vvo injection and uptake by professional antigen-presenting cells (APCs) in various tissues the CRBD-1 is expressed in APCs and displayed for the immune response.

Various modifications of mRNA may be used in order to counter the degradation of a CRBD mRNA therapeutic or vaccine disclosed herein.

In one embodiment, the mRNA comprises a 5′ cap. The 5′ cap is a specially altered nucleotide on the 5′ end of mRNA. This process, known as mRNA capping, is highly regulated and vital in the creation of stable and mature messenger RNA able to undergo translation during protein synthesis. In eukaryotes the 5′ cap consists of a guanine nucleotide connected to mRNA via an unusual 5′ to 5′ triphosphate linkage. This guanosine is methylated directly after capping in vivo by a methyltransferase. In multicellular eukaryotes further modifications exist, including the methylation of the first 2 ribose sugars of the 5′ end of the mRNA. The 5′ cap is chemically similar to the 3′ end of an RNA molecule and this provides significant resistance to 5′exonucleases. Eukaryotic RNA undergoes a series of modifications in order to be exported from the nucleus and successfully translated into function proteins, many of which are dependent on mRNA capping, the first mRNA modification to take place. Various versions of 5′ caps can be added during or after the transcription reaction using various capping enzymes such as a vaccinia virus capping enzyme or by incorporating a synthetic cap or anti-reverse cap analogues.

In another embodiment, the mRNA further comprises a poly(A) tail of between 50 and 120 contiguous adenosine residues. Polyadenylation helps protect the mRNA 3′ end against degradation by exonucleases, the export of mature mRNA to the cytoplasmic environment, and also for mRNA translation.

In another embodiment, the mRNA comprises a 5′ untranslated region comprising the sequence GGGAGACUGCCACCAUG (SEQ ID NO: 33) or GGGAGACUGCCAAGAUG (SEQ ID NO: 34) The 5-untranslated region (5′-UTR) of mRNA of this embodiment contains structural elements, which are recognized by cell-specific RNA-binding proteins, thereby affecting the translation of the molecule. To create recombinant RNA transcripts with short synthetic 5′-UTRs, the corresponding DNA sequences may be cloned into a plasmid vector upstream of the CRBD gene. Table 1 lists the positions of different bases in the mRNA relative to the start codon. T7 promoter (TAATACGACTCACTATA (SEQ ID NO: 35)) may be combined with the Kozak element consensus sequence upstream of the start codon (ATG). Transcription from T7 promoter begins with the first G after the TATA element. The following six bases after the TATA element (GGGAGA) help provide high yields and homogenous 5′mRNA ends during in vitro transcription. This template-sequence results in an RNA, which has the sequence GGGAGACUGCCA (C/A) (C/G) AUG (SEQ ID NO: 37) as its 5′-UTR.

Table 1 shows two minimal UTRs with best results as 5′-UTRs.

TABLE 1
Sequences of Synthetic 5′-Untranslated
Region

| | | Trans- | Extra | Kozak | |
|---|---|---|---|---|---|
| Minimal | | cription | nucleo- | se | Start |
| UTR | Promoter | start site | tides | quence | codon |
| UTR1 | T7 | GGGAGA | CT | GCCACC | ATG |
| UTR2 | T7 | GGGAGA | CT | GCCAAG | ATG |

In a further embodiment, the mRNA comprises a 3′ untranslated region comprising one or two copies of a beta globin mRNA 3′-UTR. Any beta globin mRNA 3′-UTR may be used as deemed suitable for an intended purpose. In one embodiment, the beta globin mRNA 3′-UTR comprises the amino acid sequence of SEQ ID NO:26.

(SEQ ID NO: 26}
GCUCGCUUUCUUGCUGUCCAAUUUCUAUUAAAGGUUCCUUUGUUCCCUAA
GUCCAACUACUAAACUGGGGGAUAUUAUGAAGGGCCUUGAGCAUCUGGAU
UCUGCCUAAUAAAAAACAUUUAUUUUCAUUGC

In another aspect, the disclosure provides pharmaceutical compositions comprising

(a) the polypeptide, the multimer, the scaffold, the nucleic acid, the recombinant expression vector, and/or the cell of an preceding claim; and

(b) a pharmaceutically acceptable carrier.

The pharmaceutical compositions of the disclosure may be used, for example, in the methods of the disclosure. In one embodiment the composition comprises the pharmaceutically acceptable carrier and:

(a) the CRBD of SEQ ID NO:3 and the CRBD of SEQ ID NO:4;

(b) the isolated polypeptide of SEQ ID NO:13 and the isolated polypeptide of SEQ ID NO:14;

(c) the CRBD of SEQ ID NO:5 and the CRBD of SEQ ID NO:6;

(d) the isolated polypeptide of SEQ ID NO: 15 and the isolated polypeptide of SEQ ID NO:16; and/or

(e) two or all three of the isolated polypeptide of SEQ ID NO:20, the isolated polypeptide of SEQ ID NO:21, and the isolated polypeptide of SEQ ID NO:32;

or multimers or scaffolds thereof.

In another embodiment, compositions comprise

(a) the mRNA of any embodiment or combination of embodiments herein; and

(b) a cationic lipid such as a liposome, or a cationic protein such as protamine.

Any cationic lipid or protein may be used as deemed appropriate for an intended use, including but not limited to liposomes and protamine. The cationic lipid or cationic protein and mRNA may be present in any suitable ratio. In one non-limiting embodiment, the cationic lipid or protein (including but not limited to protamine) and mRNA are present in a mass ratio of about 1:5, about 1:2, about 1:1 or about 2:1.

In one such embodiment, the mRNA encodes:

(a) the CRBD of SEQ ID NO:3 and the CRBD of SEQ ID NO:4;

(b) the isolated polypeptide of SEQ ID NO:13 and the isolated polypeptide of SEQ ID NO:14;

(c) the CRBD of SEQ ID NO:5 and the CRBD of SEQ ID NO:6;

(d) the isolated polypeptide of SEQ ID NO:15 and the isolated polypeptide of SEQ ID NO:16; and/or

(e) two or all three of the isolated polypeptide of SEQ ID NO:20, the isolated polypeptide of SEQ ID NO:21, and the isolated polypeptide of SEQ ID NO:32.

The pharmaceutical composition may further comprise (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptide, the multimer, the scaffold, the nucleic acid, the recombinant expression vector, and/or the cell of any embodiment or combination of embodiments herein may be the sole active agent in the pharmaceutical composition, or the composition may further comprise one or more other active agents suitable for an intended use.

In another aspect, the disclosure provides methods for treating a SARS coronavirus infection, comprising administering to a subject infected with a SARS coronavirus an amount effective to treat the infection of the polypeptide, the multimer, the scaffold, the nucleic acid, the recombinant expression vector, the cell, and/or the pharmaceutical composition of any claim herein.

As used herein, “treat” or “treating” means accomplishing one or more of the following in an individual that already has a SARS coronavirus infection: (a) reducing the severity of the infection: (b) limiting or preventing development of symptoms characteristic of the infection being treated; (c) inhibiting worsening of symptoms characteristic of the infection; and (d) limiting or preventing recurrence of symptoms in patients that were previously symptomatic for the infection.

In another aspect, the disclosure provides methods for limiting development of a SARS coronavirus infection, comprising administering to a subject at risk of SARS coronavirus infection an amount effective to limit development of a SARS coronavirus infection of the polypeptide, the multimer, the scaffold, the nucleic acid, the recombinant expression vector, the cell, and/or the pharmaceutical composition of any claim herein.

As used herein, “limiting” or “limiting development of” means accomplishing one or more of the following in an individual that does not have a SARS coronavirus infection: (a) preventing infection; (b) reducing the severity a subsequent infection; and (c) limiting or preventing development of symptoms after a subsequent infection.

In a further aspect, the disclosure provides methods for generating an immune response in a subject, comprising administering to the subject an amount effective to generate an immune response of the polypeptide, the multimer, the scaffold, the nucleic acid, the recombinant expression vector, the cell, and/or the pharmaceutical composition of any claim herein.

In this aspect, generating an immune response can be used to prevent infection, treat an existing infection or limit development of a subsequent infection.

In all of the above aspects, an “amount effective” refers to an amount of the therapeutic that is effective for treating and/or limiting the infection. The polypeptide, the multimer, the scaffold, the nucleic acid, the recombinant expression vector, the cell, and/or the pharmaceutical composition may be administered by any suitable route. In one embodiment of all of these aspect, the polypeptide, the multimer, the scaffold, the nucleic acid, the recombinant expression vector, the cell, and/or the pharmaceutical composition may be administered by subcutaneous, intradermal or intramuscular injection.

The subject in any of the methods disclosed herein may be any subject infected with or at risk or a SARS coronavirus infection, including but not limited to a human subject.

In another aspect, the disclosure provides methods for monitoring a SARS coronavirus-induced disease in a subject and/or monitoring response of the subject to immunization by a SARS coronavirus vaccine, comprising contacting the polypeptide, the multimer, the scaffold, the nucleic acid, the recombinant expression vector, the cell, and/or the pharmaceutical composition of any claim herein with a bodily fluid from the subject and detecting SARS coronavirus-binding antibodies in the bodily fluid of the subject. In this embodiment, a change in SARS coronavirus-binding antibodies in the bodily fluid of the subject can be monitored over time after the therapeutic or prophylactic methods disclosed herein, or any other therapeutic or prophylactic methods to treat or limit development of a SARS coronavirus-induced disease.

In one embodiment, the bodily fluid comprises serum or whole blood.

In a further aspect, the disclosure provides methods for detecting SARS coronavirus binding antibodies, comprising

(a) contacting the polypeptide, the multimer, the scaffold, and/or the pharmaceutical composition of any claim herein with a composition comprising a candidate SARS coronavirus binding antibody under conditions suitable for binding of SARS coronavirus antibodies to the polypeptide, the multimer, the scaffold, and/or the pharmaceutical composition; and

(b) detecting SARS coronavirus antibody complexes with the polypeptide, the multimer, the scaffold, and/or the pharmaceutical composition. In this embodiment, the reagents disclosed herein can be used in testing a subject for SARS coronavirus infection.

In one embodiment, the method further comprises isolating the SARS coronavirus antibodies that can be used, for example, as therapeutic antibodies to treat a subject having a SARS coronavirus infection.

In a further embodiment, the disclosure provides methods for producing SARS coronavirus antibodies, comprising

(a) administering to a subject an amount effective to generate an antibody response of the polypeptide, the multimer, the scaffold, the nucleic acid, the recombinant expression vector, the cell, and/or the pharmaceutical composition of any claim herein; and

(b) isolating antibodies produced by the subject. In this aspect, antibodies may be isolated and used, for example, as therapeutic antibodies to treat a subject having a SARS coronavirus infection.

### EXAMPLES

### Example 1. Characterization of Self-Assembled Nanoparticles with 60 Copies of the Polypeptide Comprising the Amino Acid Sequence of SEQ ID NO:20 by Negative-Staining Electron Microscopy

In order to confirm that the polypeptides disclosed herein comprising the multimerization domain of lumazine synthase self-assembles into a nanoparticle comprising 60 copies of the isolated polypeptide, the polypeptide of SEQ ID NO:20 is expressed and purified and the self-assembled secreted protein VX2024 is imaged by negative-staining electron microscopy.

Plasmid Construct

A construct encoding for the amino acid sequence of SEQ ID NO:20 fused with an N-terminal human IL-2 signal peptide was cloned into a pHL-sec vector (Addgene).

Expression of Polypeptide

FreeStyle™ 293 cells (2×10⁶) in 500 ml of SFM media were transfected using 600 μg of plasmid DNA according to Life Technologies protocol. After 7 days of transfection cells were harvested and spun down at 4600×g for 30 min at 4° C. Supernatant was removed from beads and filtered through 0.22 μm filter and stored at 4° C. for lectin chromatography.

Lectin Chromatography

5 ml of lectin beads (Vector Laboratories) were washed and added to 500 ml transfected supernatant and rocked over night at 4° C. Supernatant and suspended beads were allowed to flow through a disposable plastic column via gravity. The collected beads were washed with 5 column volumes of phosphate buffered saline (PBS). Elution of protein was done using lectin elution buffer (Vector Laboratories). Wash and elution were monitored by Bio-Rad UV spectrum.

Dialysis and Concentration of Protein

Protein was dialyzed overnight in PBS in dialysis cassettes. The dialyzed protein solution was spun down at 3500 RPM during 10 min, then the supernatant collected without disturbing the precipitated pellet. Concentration was done with centrifugal concentrator (Amicon Ultram 3 k MWCO UFC800396) spun at 3500 RPM for 20 min.

Size Exclusion Chromatography

500 μl of concentrated protein were passed through Superdex™ 10/300GL column using BioRad Biologic Duo™ Flow. Purified proteins were collected using first batch of concentrated protein.

SDS-PAGE and Western Blot Analysis

Protein samples were collected at different stages and samples were analyzed on a 4-12% BIS-TRIS SDS-PAGE with reducing agent. Purified VX2024 protein was further analyzed by Western blot. After transfer to PVDF membrane the membrane was incubated with a polyclonal anti-RBD antibody (Sino Biological) at 1:2000 dilution followed by incubation with HRP conjugated anti-rabbit polyclonal antibody and reaction was detected by chemiluminescence. The antibody reacted to a protein at approximately 31 kDa, which is the estimated molecular weight of the polypeptide (31.6 kDa).

Negative Stain Electron Microscopy (NS EM) Analysis

VX2024 sample was prepared in PBS at 1 mg/mi concentration and stored at 4° C. until imaging.

Sample was diluted in TBS to 20 μg/ml and applied to a carbon coated copper grid (400-mesh). Uranyl-formate was used for staining and grids were prepared in duplicates to check for reproducibility.

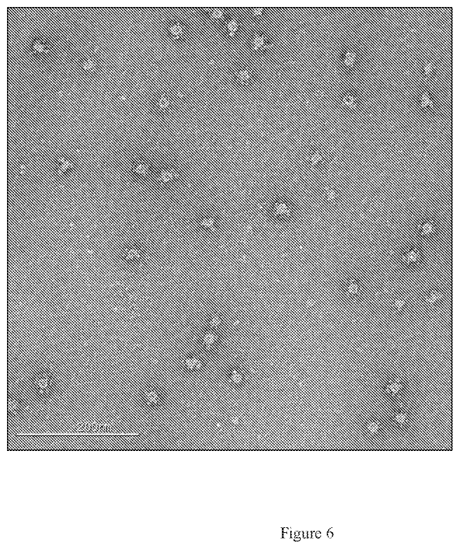

EM imaging was performed with TF20 microscope (200 kV) and Tietz 4 k/4 k camera. As shown in the images (FIG. 6) the sample was very homogeneous and particles are homogeneous in size.

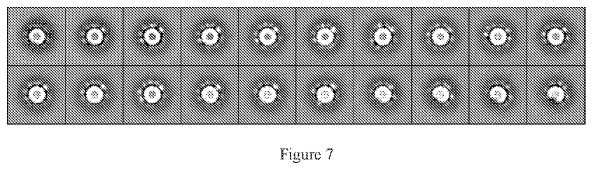

186 micrographs were acquired for duplicate grids. 3,721 particles were picked manually and 2D-aligned into 20 classes (FIG. 7). Nanoparticles appeared as expected by the design with scattered CRBD-1 antigen densities around the lumazine synthase core. The diameter of the full nanoparticle was approximately 30 nm.

### Example 2. Immunogenicity of VX2024 Vaccine Comprising the Amino Acid Sequence of SEQ ID NO:20 in Mouse

The following experiment confirms that the vaccine VX2024 with nanoparticles comprising the amino acid sequence of SEQ ID NO:20 elicits neutralizing antibodies in vaccinated mouse with inhibition of the RBD-ACE2 interaction comparable to COVID-19 human patient sera. Moreover because SEQ ID NO:20 comprises the amino acid sequence of the conserved antigen CRBD-1 this inhibition by antibodies recognizing epitopes located on the CRBD-1 is not affected by mutations of the virus occurring outside the CRBD-1 antigen. In particular the recent variants of concern of SARS-CoV-2, namely B.1.1.7 (originated in the U.K), B.1.351 (originated in the Republic of South Africa) and P.1 (originated in Brazil) incorporate the key mutations N501 Y and E484K in residues located outside the CRBD-1. These mutations increase the transmissibility and pathogenicity of the virus and also facilitate the virus escape from neutralizing antibodies recognizing epitopes comprising the residues E484 or N501. Accordingly the inhibition of the RBD-ACE2 interaction with antibodies elicited by the VX2024 vaccine, and the neutralization of the virus by these antibodies recognizing epitopes located on the CRBD-1 antigen, is independent of these mutations. Contrary to all vaccines using the Spike protein or the RBD the efficacy of the reagents disclosed herein, exemplified by VX2024, is therefore unaffected by these mutations.

Expression and Purification of Protein

The VX2024 protein was expressed and purified as described in Example 1.

Vaccine Formulation A vaccine was formulated with aluminum hydroxide wet gel suspension (Alhydrogel adjuvant 2%, InvivoGen) with 1:1 volume ratio of adjuvant to VX2024 solution and final VX2024 protein concentration of 100 μg/mL.

Vaccine was dispensed in 2 vials for mouse injection (volume 100 μL and protein dose 10 μg) at 2 different dates (week 0 and 3) and stored at room temperature (20° C.).

Immunization

N=10 CB6F1/J female mice (The Jackson Laboratory) 6-8 weeks old were dosed subcutaneously at the base of the tail with the vaccine at week 0 and 3. Dose 10 μg/100 μL.

Blood Collection

Blood was collected into clot activator tubes via submandibular vein at 200 μL per collection in week 0 (prior to dose), 2 and 5. All blood samples were allowed to clot at room temperature, centrifuged ambient (20° C.) at 3000 RPM for 15 minutes, and serum supernatant was stored frozen at −80° C.

ELISA Analysis

To determine if VX2024 elicits neutralizing antibodies we analyzed randomly selected samples of week 0 collection and all 10 samples of week 5 collection with the SARS-CoV-2 surrogate Virus Neutralization Test (sVNT) Kit (GenScript). The assay detects any antibodies in serum and plasma that neutralize the RBD-ACE2 interaction. The test is both species and isotype independent.

The SARS-CoV-2 sVNT Kit is a blocking ELISA detection tool, which mimics the virus neutralization process. The kit contains two key components: the Horseradish peroxidase (HRP) conjugated recombinant SARS-CoV-2 RBD fragment (HRP-RBD) and the human ACE2 receptor protein (hACE2). The protein-protein interaction between HRP-RBD and hACE2 is blocked by neutralizing antibodies against SARS-CoV-2 RBD.

First, the samples and controls are pre-incubated with the HRP-RBD to allow the binding of the circulating neutralization antibodies to HRP-RBD. The mixture is then added to the capture plate which is pre-coated with the human ACE2 protein. The unbound HRP-RBD as well as any HRP-RBD bound to non-neutralizing antibody is captured on the plate. while the circulating neutralization antibodies_HRP-RBD complexes remain in the supernatant and get removed during washing. After washing steps, 3,3′,5,5′-tetramethylbenzidine (TMB) solution is added, making the color blue. By adding Stop Solution, the reaction is quenched and the color turns yellow. This final solution is read at 450 nm in a microtiter plate reader. The absorbance of the sample is inversely dependent on the titer of the anti-SARS-CoV-2 neutralizing antibodies.

The inhibition rate is calculated with the net optical density (OD450) of sample and kit negative control as follows:

Inhibition=(1−OD value of sample/OD value of negative control)×100%

The positive and negative cutoff for SARS-CoV-2 neutralizing antibody detection is used for interpretation of the inhibition rate. The cutoff value of 20% is based on validation with a panel of confirmed COVID-19 patient sera and healthy control sera (GenScript).

### Results

The inhibition of week 0 samples ranged from 9.98% to 12.06% with a mean value of 11.02% indicating no detectable SARS-CoV-2 neutralizing antibody.

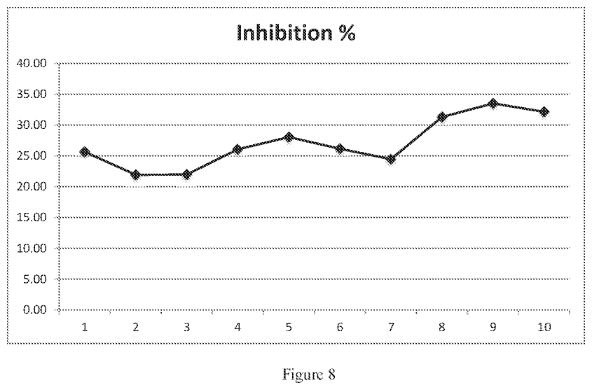

For all ten week 4 samples inhibition percentage was higher than 20% indicating detection of SARS-CoV-2 neutralizing antibodies in the mouse sera (mean 27.12, standard deviation 4.07, range 21.92 to 33.51) (FIG. 8)

Therefore the VX2024 vaccination schedule induced neutralizing antibodies in all mouse sera with inhibition of RBD-ACE2 interaction comparable to human COVID-19 patient sera and independently of mutations E484K and N501Y of variants of concern.

### Example 3. Immunogenicity in Mouse of VX2024r mRNA Vaccine Encoding for the Amino Acid Sequence of SEQ ID NO:20

The following experiments confirm that the mRNA vaccine VX2024r encoding for the amino acid sequence of SEQ ID NO:20 elicits neutralizing antibodies in vaccinated mouse with inhibition of the RBD-ACE2 interaction comparable to COVID-19 human patient sera. As described in Example 2, because SEQ ID NO:20 comprises the amino acid sequence of the conserved antigen CRBD-1, this inhibition by antibodies recognizing epitopes located on the CRBD-1 is unaffected by any mutation situated outside the CRBD-1, and more particularly the mutations N501Y and E484K of the recent variants of concern B.1.1.7, B.1.351 and P.1. Accordingly the inhibition of the RBD-ACE2 interaction with antibodies elicited by the reagents disclosed herein, exemplified by VX2024 vaccine, and the neutralization of the SARS-CoV-2 virus by these antibodies is independent of these mutations, as they facilitate the virus escape from neutralizing antibodies recognizing epitopes comprising the residues E484 or N501. Contrary to all vaccines using the Spike protein or the RBD antigen the efficacy of VX2024 is therefore unaffected by these mutations.

Construct in pUC19 Plasmid

A construct with the 5′ minimal untranslated region UTR1 of Table 1, the human IL-2 signal sequence, a nucleotide sequence encoding the amino acid sequence of SEQ ID NO:20, the 3′UTR region of SEQ ID NO:100, a poly(A) tail of 70 adenosine residues and the BsmBI restriction site was cloned into the pUC19 vector.

mRNA Transcription and Capping

The supercoiled pUC19 DNA was upscaled and linearized with the enzyme BsmBI, and in vitro transcription was performed with T7 polymerase in a 2 mL reaction. The mRNA was capped on the 5′ end with vaccinia enzymatic capping. Final yield of VX2024r mRNA was 3.27 mg after purification.

Vaccine Formulation

The mRNA VX2024r was complexed with the polycationic protein protamine by addition of protamine to the mRNA at a mass ratio of 1:5. The VX2024r vaccine was prepared on each injection day with final VX2024r mRNA concentration of 840 μg/mL.

Immunization

In a first experiment N=8 CB6F1/J female mice (The Jackson Laboratory) 6-8 weeks old were dosed by intradermal injection at the ear pinna under 1-5% isoflurane anesthesia with the vaccine at week 0 and 2. Dose 42 μg/50 μL.

In a second experiment N=8 CB6F1/J female mice 6-8 weeks old were dosed by intradermal injection at the car pinna (mouse 1-4) or by intramuscular injection at the caudal thigh (mouse 5-8) with a needle-free injection system (Tropis injector modified for mouse injection, Pharmajet) under 1-5% isoflurane anesthesia at week 0 and 2. Dose 42 μg/50 μL.

Blood Collection

Blood was collected into clot activator tubes via retro-orbital under 1-5% isoflurane anesthesia at 200 μL per collection in week 0 (prior to dose) and 4. All blood samples were allowed to clot at room temperature, centrifuged ambient (20° C.) at 3000 RPM for 15 minutes, and serum supernatant was stored frozen at −80° C.

ELISA Analysis

To determine if VX2024 elicits neutralizing antibodies we analyzed all samples of week 4 collection with the SARS-CoV-2 surrogate Virus Neutralization Test (sVNT) Kit (GenScript) described in Example 3.

The RBD-ACE2 interaction inhibition rate is calculated with the net optical density (OD450) of sample and kit negative control as follows:

Inhibition=(1−OD value of sample/OD value of negative control)×100%

The cutoff value of 20% is based on validation with a panel of confirmed COVID-19 patient sera and healthy control sera (GenScript).

### Results

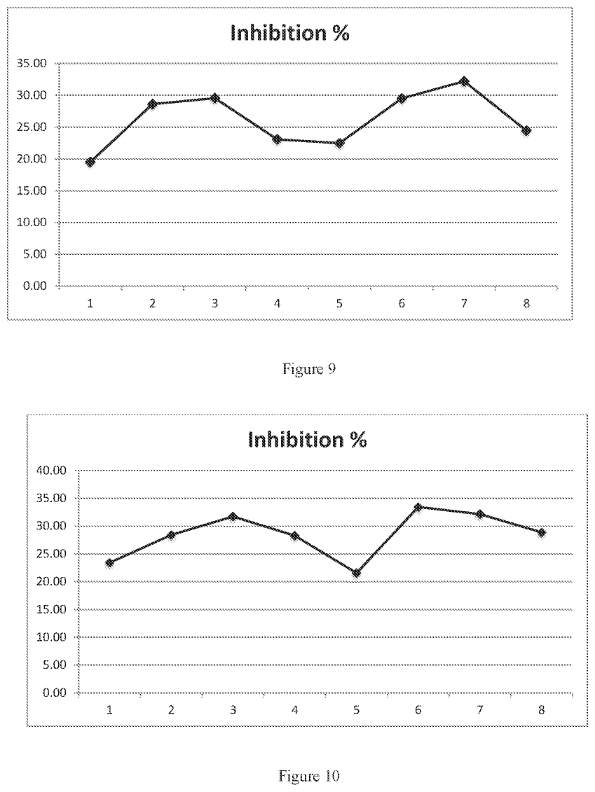

In the first experiment for 7/8 week 4 samples inhibition percentage was higher than 20% indicating detection of SARS-CoV-2 neutralizing antibodies in the mouse sera (mean 26.17, standard deviation 4.40, range 19.51 to 32.19) (FIG. 9)

In the second experiment the inhibition of week 0 samples ranged from 8.82% to 14.15% with a mean value of 10.94% indicating no detectable SARS-CoV-2 neutralizing antibody. For all 8 week 4 samples inhibition percentage was higher than 20% indicating detection of SARS-CoV-2 neutralizing antibodies in the mouse sera (mean 28.47, standard deviation 4.18, range 21.54 to 33.42) (FIG. 10). The inhibition rate with needle-free intramuscular injection was slightly improved with a mean value of 28.99% (mouse 5-8) as compared to a mean value of 27.94% with the standard ear pinna intradermal injection (mouse 1-4) (FIG. 10).

Therefore the VX2024 vaccination schedule induced neutralizing antibodies in 15/16 mouse sera with inhibition of RBD-ACE2 interaction comparable to human COVID-19 patient sera, with two different routes of administration, and independently of the mutations E484K and N501Y of SARS-CoV-2 variants of concern.

### Example 4. Immunogenicity in Mouse of VX2024rM mRNA Vaccine Encoding for the Amino Acid Sequence of SEQ ID NO:22

In order to avoid a potential cysteine mispairing and conformational epitope change the amino acid sequence of SEQ ID NO:20 was mutated in position 230 with a cysteine mutated into an alanine.

(SEQ ID NO: 32)
MQIYEGKLTAEGLRFGIVASRANHALVDRLVEGAIDAIVRHGGREEDI
TLVRVCGSWEIPVAAGELARKEDIDAVIAIGVLCRGATPSFDYIASEV
SKGLADLSLELRKPITFGVITADTLEQAIEAAGTCHGNKGWEAALCAI
EMANLFKSLRGGSGGSGGSGGSGGGNITNLCPFGEVFNATRFASVYAW
NRKRISNCVADYSVLYNSASFSTFKCYGVSPTKLNDLAFTNVYADSFV
IRGDEVRQIAPGQTGKIADYNYKLPDDFTGCVIAWN

The following experiment confirms that the mRNA vaccine VX2024rM encoding for the amino acid sequence of SEQ ID NO:22 elicits neutralizing antibodies in vaccinated mouse with inhibition of the RBD-ACE2 interaction comparable to COVID-19 human patient sera, and independently of the mutations E484K and N501Y of SARS-CoV-2 variants of concern.

Construct in pUC19 μPlasmid

A construct with the 5′ minimal untranslated region UTR1 of Table 1, the human IL-2 signal sequence, a nucleotide sequence encoding the amino acid sequence of SEQ ID NO:22, the 3′UTR region of SEQ ID NO:100, a poly(A) tail of 70 adenosine residues and the BsmBI restriction site was cloned into pUC19 vector.

mRNA Transcription and Capping

The supercoiled pUC19 DNA was upscaled and linearized with the enzyme BsmBI, and in vitro transcription was performed with T7 polymerase in a 2 mL reaction. The mRNA was capped on the 5′ end with vaccinia enzymatic capping. Final yield of VX2024rM mRNA was 5.14 mg after purification.

Vaccine Formulation

The mRNA VX2024rM was complexed with the polycationic protein protamine by addition of protamine to the mRNA at a mass ratio of 1:5. The VX2024rM vaccine was prepared on each injection day with final VX2024rM mRNA concentration of 840 μg/mL.

Immunization

N=8 CB6F1/J female mice (The Jackson Laboratory) 6-8 weeks old were dosed by intradennal injection at the ear pinna (mouse 1-4) or by intramuscular injection at the caudal thigh (mouse 5-8) with a needle-free injection system (Tropis™ injector modified for mouse injection. PharmaJet) under 1-5% isoflurane anesthesia at week 0 and 2. Dose 42 μg/50 μL.

Blood Collection

Blood was collected into clot activator tubes via retro-orbital under 1-5% isoflurane anesthesia at 200 μL per collection in week 0 (prior to dose) and 4. All blood samples were allowed to clot at room temperature, centrifuged ambient (20° C.) at 3000 RPM for 15 minutes, and serum supernatant was stored frozen at −80° C.

ELISA Analysis

To determine if VX2024rM elicits neutralizing antibodies we analyzed all 8 samples of week 4 collection with the SARS-CoV-2 surrogate Virus Neutralization Test (sVNT) Kit (GenScript) described in Example 3.

The RBD-ACE2 interaction inhibition rate is calculated with the net optical density (OD450) of sample and kit negative control as follows:

Inhibition=(1−OD value of sample/OD value of negative control)×100%

The cutoff value of 20% is based on validation with a panel of confirmed COVID-19 patient sera and healthy control sera (GenScript).

### Results

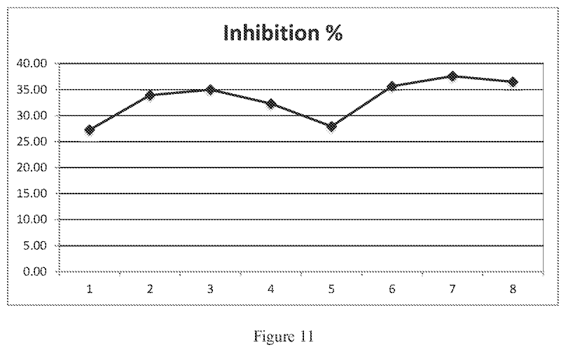

For all 8 week 4 samples inhibition percentage was higher than 20% indicating detection of SARS-CoV-2 neutralizing antibodies in the mouse sera (mean 33.25, standard deviation 3.85, range 27.24 to 37.57) (FIG. 9) The inhibition rate with needle-free intramuscular injection was improved with a mean value of 34.40% (mouse 1-4) as compared to a mean value of 32.11% with the standard ear pinna intradermal injection (mouse 5-8) (FIG. 11).

The RBD-ACE2 interaction inhibition rate with the VX2024rM vaccine was improved as compared with the VX2024r vaccine of Example 3 for both routes of administration (mean 32.11 vs 27.94 for car pinna i.d. and 34.40 vs 28.99 for needle-free i.m., standard deviation 3.43 vs 3.42 for ear pinna i.d. and 4.40 vs 5.32 for needle-free i.m.)

Therefore the VX2024rM vaccination schedule induced neutralizing antibodies in all 8 mouse sera with inhibition of RBD-ACE2 interaction comparable to human COVID-19 patient sera, with two different routes of administration, with an advantage for needle-free intramuscular injection, and independently of the mutations E484K and N501Y of SARS-CoV-2 variants of concern.

1. An isolated polypeptide comprising a conserved receptor-binding domain (CRBD) from a severe acute respiratory syndrome (SARS) coronavirus spike protein, wherein the CRBD comprises an amino acid sequence at least 70% identical to the amino acid sequence selected from the group consisting of SEQ ID NOS:1-9, wherein the polypeptide includes no more than 10 amino acid residues from a variable receptor binding motif (VRBM) from a SARS coronavirus spike protein, or includes no amino acid residues from the VRBM of a SARS coronavirus spike protein.

2. The isolated polypeptide of claim 1, wherein the CRBD comprises an amino acid sequence at least 70% identical to the amino acid sequence selected from the group consisting of SEQ ID NOS: 1-8, 10, 22, and 38.

3. (canceled)

4. The isolated polypeptide of claim 2, wherein differences in the CRBD amino acid sequence and SEQ ID NO:10 comprise differences at 1 or more positions selected from residues 11, 14, 15, 16, 18, 24, 27, 34, 37, 42, 43, 48, 54, 63, 72, 73, 76, 79, 87, 100, 104, and 105 in SEQ ID NO:10 or 22.

5. The isolated polypeptide of claim 2, wherein the CRBD comprises:
(a) the amino acid sequence of SEQ ID NO:1, wherein at least 1 of the residues bounded by parentheses is the first listed residue:
(SEQ ID NO: 1)
NITNLCPFGE (V/I) FN (A/S) (T/S) (R/T/K) FASVYAW
(N/D) RKRISNCVA (D/Y) YS (V/F) LYNS (A/T) SFSTF
(K/R) CYGVSPTKLNDLCFTNVYADSFV (I/V) (R/T/K)
GDEVR (Q/E) IAPGQTG (K/R/V) IADYNYKLPDDFTGCVI
(A/S) WN;
(b) the amino acid sequence of SEQ ID NO:2, wherein at least 1 of the residues bounded by parentheses is the first listed residue:
(SEQ ID NO: 2)
NITNLCPFGEVFNA (T/S) (R/T/K) FASVYAWNRKRISNCV
ADYSVLYNS (A/T) SFSTFKCYGVSPTKLNDLCFTNVYADSFV
(I/V) (R/T/K) GDEVRQIAPGQTG (K/R/V) IADYNYKLP
DDFTGCVIAWN;
(c) the amino acid sequence of SEQ ID NO:3 or SEQ ID NO:4, wherein 1 of the residues bounded by parentheses is the first listed residue:
(SEQ ID NO: 3)
NITNLCPFGE (V/I) FN (A/S) (T/S) (R/T/K) F (A/P)
SVYAW (N/E/D) RK (R/K)ISNCVA (D/Y) YS (V/F)
LYNSA (S/F) FSTF (K/R) CYGVS (P/A) TKLNDLCF
(T/S) NVYADSFV (I/V) (R/T/K) GD (E/D) VR (Q/E)
IAPGQTG (K/R/V)IADYNYKLPDDF (T/M) GCV (I/L)
(A/S) WN
(SEQ ID NO: 4)
NITNLCPFGE (V/I) FN (A/S) (T/S) (R/T/K) F (A/P)
SVYAW (N/E/D) RK (R/K) ISNCVA (D/Y) YS (V/F)
LYNST (S/F) FSTF (K/R) CYGVS (P/A) TKLNDLCF
(T/S) NVYADSFV (I/V) (R/T/K) GD (E/D) VR (Q/E)
IAPGQTG (K/R/V)IADYNYKLPDDF (T/M) GCV (I/L)
(A/S) WN;
(d) the amino acid sequence of SEQ ID NO:5 or SEQ ID NO:6, wherein at least 1 of the residues bounded by parentheses is the first listed residue:
(SEQ ID NO: 5)
NITNLCPFGEVFNA (T/S) (R/T/K) FASVYAWNRKRISNCVA
DYSVLYNSASFSTFKCYGVSPTKLNDLCFTNVYADSFV (I/V)
(R/T/K) GDEVRQIAPGQTG (K/R/V) IADYNYKLPDDFTGCV
IAWN
(SEQ ID NO: 6)
NITNLCPFGEVFNA (T/S) (R/T/K) FASVYAWNRKRISNCVA
DYSVLYNSTSFSTFKCYGVSPTKLNDLCFTNVYADSFV
(I/V) (R/T/K) GDEVRQIAPGQTG (K/R/V) IADYNYKLPD
DFTGCVIAWN;
(e) the amino acid sequence of SEQ ID NO:7, wherein at least 1 of the amino acid residues in SEQ ID NO:7 bounded by parentheses are the first listed residue
(SEQ ID NO: 7)
NITNLCPFGEVFNAT (R/K) F (A/P) SVYAW (N/E) RK
(R/K) ISNCVADYSVLYNS (A/T)(S/F) FSTFKCYGVS
(P/A) TKLNDLCF (T/S) NVYADSFV (I/V) (R/K) GD
(E/D) VRQIAPGQTG (K/V) IADYNYKLPDDF (T/M) GCV
(I/L) AWN;
(f) the amino acid sequence of SEQ ID NO:8, wherein at least 1 of the residues bounded by parentheses is the first listed residue.
(SEQ ID NO: 8)
NITNLCPFGE (V/I) FN (A/S) (T/S) (R/T/K) F (A/P)
SVYAW (N/E/D) RK (R/K)ISNCVA(D/Y) YS (V/F) LYNS
(A/T) (S/F) FSTF (K/R) CYGVS (P/A) TKLNDLCF
(T/S) NVYADSFV (I/V)(R/T/K) GD (E/D) VR (Q/E)
IAPGQTG (K/R/V) IADYNYKLPDDF (T/M) GCV (I/L)
(A/S) WN

6.-10. (canceled)

11. The isolated polypeptide of claim 1, further comprising a multimerization domain.

12. The isolated polypeptide of claim 11, wherein the multimerization domain comprises an amino acid sequence at least at least 70% identical to the amino acid sequence of SEQ ID NO: 23, 24, 27, or 28.

13.-17. (canceled)

18. The isolated polypeptide of claim 11 comprising the amino acid sequence selected from the group consisting of SEQ ID NOS: 11-18 and 39, wherein n is 3-7, 3-6, 3-5, 3-4, 4-7, 4-6, 4-5, 5-7, 5-6, 3, 4, 5, 6, or 7, or comprising the amino acid sequence of SEQ ID NO:20, SEQ ID NO:21, or SEQ ID NO:22.

19. (canceled)

20. The isolated polypeptide of claim 11, comprising a general formula selected from the group consisting of: X1-(GGS)ₙGGG-X3 and X1-(GGS)ₙGGG-X3-(GGS)ₙGGG-X2, wherein
X1 and X2 independently comprise the amino acid sequence selected from the group consisting of SEQ ID NOS:1-10, 22, and 25;
n is 3-5, 3-4, 4-5, 3, 4, 5 and
X3 comprises the amino acid sequence selected from the group consisting of SEQ ID NO:27 or 28.

21. (canceled)

22. The isolated polypeptide of claim 20, wherein X1 and X2 (when present) independently comprise the amino acid sequence selected from the group consisting of SEQ ID NOS:1-8, 10, 22, and 39.

23.-25. (canceled)

26. A multimer, comprising two or more copies of the isolated polypeptide of claim 1.

27. (canceled)

28. A scaffold, comprising two or more isolated polypeptides of claim 1 on a surface of the scaffold.

29.-31. (canceled)

32. A nucleic acid encoding the isolated polypeptide of claim 1.

33. A recombinant expression vector comprising the nucleic acid of claim 32 operatively linked to a suitable control sequence.

34. A recombinant host cell comprising the recombinant expression vector of claim 33.

35.-40. (canceled)

41. A pharmaceutical composition comprising
(a) the polypeptide of claim 1; and
(b) a pharmaceutically acceptable carrier.

42.-44. (canceled)

45. A method for treating a SARS coronavirus infection, limiting development of a SARS coronavirus infection, or generating an immune response in a subject, comprising administering to a subject in need thereof an amount effective of the polypeptide of claim 1.

46.-48. (canceled)

49. A method for monitoring a SARS coronavirus-induced disease in a subject and/or monitoring response of the subject to immunization by a SARS coronavirus vaccine, comprising contacting the polypeptide of claim 1 with a bodily fluid from the subject and detecting SARS coronavirus-binding antibodies in the bodily fluid of the subject.

50. (canceled)

51. A method for detecting SARS coronavirus binding antibodies, comprising
(a) contacting the polypeptide of claim 1 with a composition comprising a candidate SARS coronavirus binding antibody under conditions suitable for binding of SARS coronavirus antibodies to the polypeptide; and
(b) detecting SARS coronavirus antibody complexes with the polypeptide.

52. (canceled)

53. A method for producing SARS coronavirus antibodies, comprising
(a) administering to a subject an amount effective to generate an antibody response of the polypeptide of claim 1; and
(b) isolating antibodies produced by the subject.


SEQUENCE LISTING (62 sequences)

SEQ ID NO: 1 - protein, 107 aa, Artificial Sequence
  Synthetic
  MISC_FEATURE (11)..(11): X is V or I
  MISC_FEATURE (14)..(14): X is A or S
  MISC_FEATURE (15)..(15): X is T or S
  MISC_FEATURE (16)..(16): X is R, T or K
  MISC_FEATURE (24)..(24): X is N or D
  MISC_FEATURE (34)..(34): X is Y or D
  MISC_FEATURE (37)..(37): X is V or F
  MISC_FEATURE (42)..(42): X is A or T
  MISC_FEATURE (48)..(48): X is K or R
  MISC_FEATURE (72)..(72): X is I or V
  MISC_FEATURE (73)..(73): X is R, T or  K
  MISC_FEATURE (79)..(79): X is Q or E
  MISC_FEATURE (87)..(87): X is K, R or V
  MISC_FEATURE (105)..(105): X is A or S
NITNLCPFGEXFNXXXFASVYAWXRKRISNCVAXYSXLYNSXSFSTFXCYGVSPTKLNDLCFTNVYADSFVXXGDEVRXIAPGQTGXIADYNYKLPDDFTGCVIXWN

SEQ ID NO: 2 - protein, 107 aa, Artificial Sequence
  Synthetic
  MISC_FEATURE (15)..(15): X is T or S
  MISC_FEATURE (16)..(16): X is R, T or K
  MISC_FEATURE (42)..(42): X is T or A
  MISC_FEATURE (72)..(72): X is I or V
  MISC_FEATURE (73)..(73): X is R, T or K
  MISC_FEATURE (87)..(87): X is R, V or K
NITNLCPFGEVFNAXXFASVYAWNRKRISNCVADYSVLYNSXSFSTFKCYGVSPTKLNDLCFTNVYADSFVXXGDEVRQIAPGQTGXIADYNYKLPDDFTGCVIAWN

SEQ ID NO: 3 - protein, 107 aa, Artificial Sequence
  Synthetic
  MISC_FEATURE (11)..(11): X is V or I
  MISC_FEATURE (14)..(14): X is A or S
  MISC_FEATURE (15)..(15): X is T or S
  MISC_FEATURE (16)..(16): X is R, T or K
  MISC_FEATURE (18)..(18): X is A or P
  MISC_FEATURE (24)..(24): X is N, E or D
  MISC_FEATURE (27)..(27): X is R or K
  MISC_FEATURE (34)..(34): X is D or Y
  MISC_FEATURE (37)..(37): X is V or F
  MISC_FEATURE (43)..(43): X is S or F
  MISC_FEATURE (48)..(48): X is K or R
  MISC_FEATURE (54)..(54): X is P or A
  MISC_FEATURE (63)..(63): X is T or S
  MISC_FEATURE (72)..(72): X is I or V
  MISC_FEATURE (73)..(73): X is R, T or K
  MISC_FEATURE (76)..(76): X is E or D
  MISC_FEATURE (79)..(79): X is E or Q
  MISC_FEATURE (87)..(87): X is K, R or V
  MISC_FEATURE (100)..(100): X is T or M
  MISC_FEATURE (104)..(104): X is I or L
  MISC_FEATURE (105)..(105): X is A or S
NITNLCPFGEXFNXXXFXSVYAWXRKXISNCVAXYSXLYNSAXFSTFXCYGVSXTKLNDLCFXNVYADSFVXXGDXVRXIAPGQTGXIADYNYKLPDDFXGCVXXWN

SEQ ID NO: 4 - protein, 107 aa, Artificial Sequence
  Synthetic
  MISC_FEATURE (11)..(11): X is V or I
  MISC_FEATURE (14)..(14): X is A or S
  MISC_FEATURE (15)..(15): X is T or S
  MISC_FEATURE (16)..(16): X is R, T or K
  MISC_FEATURE (18)..(18): X is A or P
  MISC_FEATURE (24)..(24): X is N, E or D
  MISC_FEATURE (27)..(27): X is R or K
  MISC_FEATURE (34)..(34): X is D or Y
  MISC_FEATURE (37)..(37): X is V or F
  MISC_FEATURE (43)..(43): X is S or F
  MISC_FEATURE (48)..(48): X is K or R
  MISC_FEATURE (54)..(54): X is P or A
  MISC_FEATURE (63)..(63): X is T or S
  MISC_FEATURE (72)..(72): X is I or V
  MISC_FEATURE (73)..(73): X is R, T or K
  MISC_FEATURE (76)..(76): X is E or D
  MISC_FEATURE (79)..(79): X is E or Q
  MISC_FEATURE (87)..(87): X is K, R or V
  MISC_FEATURE (100)..(100): X is T or M
  MISC_FEATURE (104)..(104): X is I or L
  MISC_FEATURE (105)..(105): X is A or S
NITNLCPFGEXFNXXXFXSVYAWXRKXISNCVAXYSXLYNSTXFSTFXCYGVSXTKLNDLCFXNVYADSFVXXGDXVRXIAPGQTGXIADYNYKLPDDFXGCVXXWN

SEQ ID NO: 5 - protein, 107 aa, Artificial Sequence
  Synthetic
  MISC_FEATURE (15)..(15): X is T or S
  MISC_FEATURE (16)..(16): X is R, T or K
  MISC_FEATURE (72)..(72): X is I or V
  MISC_FEATURE (73)..(73): X is R, T or K
  MISC_FEATURE (87)..(87): X is R, V or K
NITNLCPFGEVFNAXXFASVYAWNRKRISNCVADYSVLYNSASFSTFKCYGVSPTKLNDLCFTNVYADSFVXXGDEVRQIAPGQTGXIADYNYKLPDDFTGCVIAWN

SEQ ID NO: 6 - protein, 107 aa, Artificial Sequence
  Synthetic
  MISC_FEATURE (15)..(15): X is T or S
  MISC_FEATURE (16)..(16): X is T, R, or K
  MISC_FEATURE (72)..(72): X is I or V
  MISC_FEATURE (73)..(73): X is R, T or K
  MISC_FEATURE (87)..(87): X is R, V or K
NITNLCPFGEVFNAXXFASVYAWNRKRISNCVADYSVLYNSTSFSTFKCYGVSPTKLNDLCFTNVYADSFVXXGDEVRQIAPGQTGXIADYNYKLPDDFTGCVIAWN

SEQ ID NO: 7 - protein, 107 aa, Artificial Sequence
  Synthetic
  MISC_FEATURE (16)..(16): X is R or K
  MISC_FEATURE (18)..(18): X is A or P
  MISC_FEATURE (24)..(24): X is N or E
  MISC_FEATURE (27)..(27): X is R or K
  MISC_FEATURE (42)..(42): X is A or T
  MISC_FEATURE (43)..(43): X is S or F
  MISC_FEATURE (54)..(54): X is P or A
  MISC_FEATURE (63)..(63): X is T or S
  MISC_FEATURE (72)..(72): X is I or V
  MISC_FEATURE (73)..(73): X is R or K
  MISC_FEATURE (76)..(76): X is E or D
  MISC_FEATURE (87)..(87): X is K or V
  MISC_FEATURE (100)..(100): X is T or M
  MISC_FEATURE (104)..(104): X is I or L
NITNLCPFGEVFNATXFXSVYAWXRKXISNCVADYSVLYNSXXFSTFKCYGVSXTKLNDLCFXNVYADSFVXXGDXVRQIAPGQTGXIADYNYKLPDDFXGCVXAWN

SEQ ID NO: 8 - protein, 107 aa, Artificial Sequence
  Synthetic
  MISC_FEATURE (11)..(11): X is V or I
  MISC_FEATURE (14)..(15): X is A or S
  MISC_FEATURE (15)..(15): X is T or S
  MISC_FEATURE (16)..(16): X is R, T or K
  MISC_FEATURE (18)..(18): X is A or P
  MISC_FEATURE (24)..(24): X is N, E or D
  MISC_FEATURE (27)..(27): X is R or K
  MISC_FEATURE (34)..(34): X is D or Y
  MISC_FEATURE (37)..(37): X is V or F
  MISC_FEATURE (37)..(37): X is V or F
  MISC_FEATURE (42)..(42): X is A or T
  MISC_FEATURE (43)..(43): X is S or F
  MISC_FEATURE (43)..(43): X is S or F
  MISC_FEATURE (48)..(48): X is K or R
  MISC_FEATURE (54)..(54): X is P or A
  MISC_FEATURE (63)..(63): X is T or S
  MISC_FEATURE (72)..(72): X is I or V
  MISC_FEATURE (73)..(73): X is R, T or K
  MISC_FEATURE (76)..(76): X is E or D
  MISC_FEATURE (79)..(79): X is E or Q
  MISC_FEATURE (87)..(87): X is K, R or V
  MISC_FEATURE (100)..(100): X is T or M
  MISC_FEATURE (104)..(104): X is I or L
  MISC_FEATURE (105)..(105): X is A or S
NITNLCPFGEXFNXXXFXSVYAWXRKXISNCVAXYSXLYNSXXFSTFXCYGVSXTKLNDLCFXNVYADSFVXXGDXVRXIAPGQTGXIADYNYKLPDDFXGCVXXWN

SEQ ID NO: 9 - protein, 21 aa, Artificial Sequence
  Synthetic
  MISC_FEATURE (16)..(16): X is H or N
GYQPYRVVVLSFELLXAPATV

SEQ ID NO: 10 - protein, 107 aa, Artificial Sequence
  Synthetic
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWN

SEQ ID NO: 11 - protein, 267 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of n repeats of (GGS)
  MISC_FEATURE (157)..(157): optional addition of “n” repeats of (GGS)
  MISC_FEATURE (171)..(171): X is V or I
  MISC_FEATURE (174)..(174): X is A or S
  MISC_FEATURE (175)..(175): X is T or S
  MISC_FEATURE (176)..(176): X is R, T or K
  MISC_FEATURE (184)..(184): X is N or D
  MISC_FEATURE (194)..(194): X is Y or D
  MISC_FEATURE (197)..(197): X is V or F
  MISC_FEATURE (202)..(202): X is A or T
  MISC_FEATURE (208)..(208): X is K or R
  MISC_FEATURE (232)..(232): X is I or V
  MISC_FEATURE (233)..(233): X is R, T, or K
  MISC_FEATURE (239)..(239): X is Q or E
  MISC_FEATURE (247)..(247): X is K, R or V
  MISC_FEATURE (265)..(265): X is A or S
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEXFNXXXFASVYAWXRKRISNCVAXYSXLYNSXSFSTFXCYGVSPTKLNDLCFTNVYADSFVXXGDEVRXIAPGQTGXIADYNYKLPDDFTGCVIXWN

SEQ ID NO: 12 - protein, 267 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of “n” repeats of (GGS)
  MISC_FEATURE (175)..(175): X is S or T
  MISC_FEATURE (176)..(176): X is R, K or T
  MISC_FEATURE (202)..(202): X is A or T
  MISC_FEATURE (232)..(232): X is I or V
  MISC_FEATURE (233)..(233): X is R, T or K
  MISC_FEATURE (247)..(247): X is R, V or K
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEVFNAXXFASVYAWNRKRISNCVADYSVLYNSXSFSTFKCYGVSPTKLNDLCFTNVYADSFVXXGDEVRQIAPGQTGXIADYNYKLPDDFTGCVIAWN

SEQ ID NO: 13 - protein, 267 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of “n” repeats of (GGS)
  MISC_FEATURE (171)..(171): X is V or I
  MISC_FEATURE (174)..(174): X is A or S
  MISC_FEATURE (175)..(175): X is T or S
  MISC_FEATURE (176)..(176): X is T, R, or K
  MISC_FEATURE (178)..(178): X is A or P
  MISC_FEATURE (184)..(184): X is N, E or D
  MISC_FEATURE (187)..(187): X is R or K
  MISC_FEATURE (194)..(194): X is D or Y
  MISC_FEATURE (197)..(197): X is V or F
  MISC_FEATURE (203)..(203): X is S or F
  MISC_FEATURE (208)..(208): X is K or R
  MISC_FEATURE (214)..(214): X is P or A
  MISC_FEATURE (223)..(223): X is T or S
  MISC_FEATURE (232)..(232): X is I or V
  MISC_FEATURE (233)..(233): X is R, T or K
  MISC_FEATURE (236)..(236): X is D or E
  MISC_FEATURE (239)..(239): X is Q or E
  MISC_FEATURE (247)..(247): X is K, R or V
  MISC_FEATURE (260)..(260): X is T or M
  MISC_FEATURE (264)..(264): X is I or L
  MISC_FEATURE (265)..(265): X is A or S
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEXFNXXXFXSVYAWXRKXISNCVAXYSXLYNSAXFSTFXCYGVSXTKLNDLCFXNVYADSFVXXGDXVRXIAPGQTGXIADYNYKLPDDFXGCVXXWN

SEQ ID NO: 14 - protein, 267 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of “n” repeats of (GGS)
  MISC_FEATURE (171)..(171): X is V or I
  MISC_FEATURE (174)..(174): X is A or S
  MISC_FEATURE (175)..(175): X is T or S
  MISC_FEATURE (176)..(176): X is R, T or K
  MISC_FEATURE (178)..(178): X is A or P
  MISC_FEATURE (184)..(184): X is N, E,  or D
  MISC_FEATURE (187)..(187): X is R  or K
  MISC_FEATURE (194)..(194): X is D  or Y
  MISC_FEATURE (197)..(197): X is V or F
  MISC_FEATURE (203)..(203): X is S or F
  MISC_FEATURE (208)..(208): X is K or R
  MISC_FEATURE (214)..(214): X is P or A
  MISC_FEATURE (223)..(223): X is T or S
  MISC_FEATURE (232)..(232): X is I or V
  MISC_FEATURE (233)..(233): X is R, T or K
  MISC_FEATURE (236)..(236): X is E or D
  MISC_FEATURE (239)..(239): X is Q or D
  MISC_FEATURE (247)..(247): X is K, R, or V
  MISC_FEATURE (260)..(260): X is T or M
  MISC_FEATURE (264)..(264): X is I or L
  MISC_FEATURE (265)..(265): X is A or S
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEXFNXXXFXSVYAWXRKXISNCVAXYSXLYNSTXFSTFXCYGVSXTKLNDLCFXNVYADSFVXXGDXVRXIAPGQTGXIADYNYKLPDDFXGCVXXWN

SEQ ID NO: 15 - protein, 267 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of “n” repeats of (GGS)
  MISC_FEATURE (175)..(175): X is S or T
  MISC_FEATURE (176)..(176): X is R, K or T
  MISC_FEATURE (232)..(232): X is I or V
  MISC_FEATURE (233)..(233): X is R, T or K
  MISC_FEATURE (247)..(247): X is R, V or K
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEVFNAXXFASVYAWNRKRISNCVADYSVLYNSASFSTFKCYGVSPTKLNDLCFTNVYADSFVXXGDEVRQIAPGQTGXIADYNYKLPDDFTGCVIAWN

SEQ ID NO: 16 - protein, 267 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of “n” repeats of (GGS)
  MISC_FEATURE (175)..(175): X is T or S
  MISC_FEATURE (176)..(176): X is R, T or K
  MISC_FEATURE (232)..(232): X is I or V
  MISC_FEATURE (233)..(233): X is R, T or K
  MISC_FEATURE (247)..(247): X is R, V or K
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEVFNAXXFASVYAWNRKRISNCVADYSVLYNSTSFSTFKCYGVSPTKLNDLCFTNVYADSFVXXGDEVRQIAPGQTGXIADYNYKLPDDFTGCVIAWN

SEQ ID NO: 17 - protein, 267 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of “n” repeats of (GGS)
  MISC_FEATURE (176)..(176): X is R or K
  MISC_FEATURE (178)..(178): X is A or P
  MISC_FEATURE (184)..(184): X is N or E
  MISC_FEATURE (187)..(187): X is R or K
  MISC_FEATURE (202)..(202): X is A or T
  MISC_FEATURE (203)..(203): X is S or F
  MISC_FEATURE (214)..(214): X is P or A
  MISC_FEATURE (223)..(223): X is T or S
  MISC_FEATURE (232)..(232): X is I or V
  MISC_FEATURE (233)..(233): X is R or K
  MISC_FEATURE (236)..(236): X is D or E
  MISC_FEATURE (247)..(247): X is K or V
  MISC_FEATURE (260)..(260): X is T or M
  MISC_FEATURE (264)..(264): X is I or L
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEVFNATXFXSVYAWXRKXISNCVADYSVLYNSXXFSTFKCYGVSXTKLNDLCFXNVYADSFVXXGDXVRQIAPGQTGXIADYNYKLPDDFXGCVXAWN

SEQ ID NO: 18 - protein, 181 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of “n” repeats of (GGS)
  MISC_FEATURE (176)..(176): X is H or N
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGGYQPYRVVVLSFELLXAPATV

SEQ ID NO: 19 - protein, 267 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of “n” repeats of (GGS)
  MISC_FEATURE (171)..(171): X is V or I
  MISC_FEATURE (174)..(174): X is A or S
  MISC_FEATURE (175)..(175): X is T or S
  MISC_FEATURE (176)..(176): X is R, T or K
  MISC_FEATURE (178)..(178): X is A or P
  MISC_FEATURE (184)..(184): X is N, E or D
  MISC_FEATURE (187)..(187): X is R or K
  MISC_FEATURE (194)..(194): X is D or Y
  MISC_FEATURE (197)..(197): X is F or V
  MISC_FEATURE (202)..(202): X is A or T
  MISC_FEATURE (203)..(203): X is S or F
  MISC_FEATURE (208)..(208): X is K or R
  MISC_FEATURE (214)..(214): X is P or A
  MISC_FEATURE (223)..(223): X is T or S
  MISC_FEATURE (232)..(232): X is I or V
  MISC_FEATURE (233)..(233): X is R, T or K
  MISC_FEATURE (236)..(236): X is E or D
  MISC_FEATURE (239)..(239): X is Q or E
  MISC_FEATURE (247)..(247): X is K, R or V
  MISC_FEATURE (260)..(260): X is T or M
  MISC_FEATURE (264)..(264): X is I or L
  MISC_FEATURE (265)..(265): X is A or S
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEXFNXXXFXSVYAWXRKXISNCVAXYSXLYNSXXFSTFXCYGVSXTKLNDLCFXNVYADSFVXXGDXVRXIAPGQTGXIADYNYKLPDDFXGCVXXWN

SEQ ID NO: 20 - protein, 276 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWN

SEQ ID NO: 21 - protein, 276 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRKRISNCVADYSVLYNSTSFSTFKCYGVSPTKLNDLCFTNVYADSFVIRGDEVRQIAPGQTGKIADYNYKLPDDFTGCVIAWN

SEQ ID NO: 22 - protein, 107 aa, Artificial Sequence
  Synthetic
NITNLCPFGEVFNATRFASVYAWNRKRISNCVADYSVLYNSTSFSTFKCYGVSPTKLNDLCFTNVYADSFVIRGDEVRQIAPGQTGKIADYNYKLPDDFTGCVIAWN

SEQ ID NO: 23 - protein, 154 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLR

SEQ ID NO: 24 - protein, 154 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLR

SEQ ID NO: 25 - protein, 20 aa, Artificial Sequence
  Synthetic
YQPYRVVVLSFELLHAPATV

SEQ ID NO: 26 - RNA, 132 nt, Artificial Sequence
  Synthetic
gcucgcuuucuugcuguccaauuucuauuaaagguuccuuuguucccuaaguccaacuacuaaacugggggauauuaugaagggccuugagcaucuggauucugccuaauaaaaaacauuuauuuucauugc

SEQ ID NO: 27 - protein, 33 aa, Artificial Sequence
  Synthetic
RMKQIEDKIEEILSKIYHIENEIARIKKLIGER

SEQ ID NO: 28 - protein, 34 aa, Artificial Sequence
  Synthetic
MKVKQLEDVVEELLSVNYHLENVVARLKKLVGER

SEQ ID NO: 29 - protein, 272 aa, Artificial Sequence
  Synthetic
  MISC_FEATURE (15)..(15): X is T or S
  MISC_FEATURE (16)..(16): X is R, T or K
  MISC_FEATURE (42)..(42): X is T or A
  MISC_FEATURE (72)..(72): X is I or V
  MISC_FEATURE (73)..(73): X is R, T or K
  MISC_FEATURE (87)..(87): X is R, V or K
  MISC_FEATURE (180)..(180): X is T or S
  MISC_FEATURE (181)..(181): X is R, T or K
  MISC_FEATURE (207)..(207): X is T or A
  MISC_FEATURE (237)..(237): X is I or V
  MISC_FEATURE (238)..(238): X is R, T or K
  MISC_FEATURE (252)..(252): X is R, V or K
NITNLCPFGEVFNAXXFASVYAWNRKRISNCVADYSVLYNSXSFSTFKCYGVSPTKLNDLCFTNVYADSFVXXGDEVRQIAPGQTGXIADYNYKLPDDFTGCVIAWNGGSGGSGGSGGGMKVKQLEDVVEELLSVNYHLENVVARLKKLVGERGGSGGSGGSGGGNITNLCPFGEVFNAXXFASVYAWNRKRISNCVADYSVLYNSXSFSTFKCYGVSPTKLNDLCFTNVYADSFVXXGDEVRQIAPGQTGXIADYNYKLPDDFTGCVIAWN

SEQ ID NO: 30 - protein, 272 aa, Artificial Sequence
  Synthetic
  MISC_FEATURE (15)..(15): X is T or S
  MISC_FEATURE (16)..(16): X is R, T or K
  MISC_FEATURE (72)..(72): X is I or V
  MISC_FEATURE (73)..(73): X is R, T or K
  MISC_FEATURE (87)..(87): X is R, V or K
  MISC_FEATURE (180)..(180): X is T or S
  MISC_FEATURE (181)..(181): X is R, T or K
  MISC_FEATURE (237)..(237): X is I or V
  MISC_FEATURE (238)..(238): X is R, T or K
  MISC_FEATURE (252)..(252): X is R, V or K
NITNLCPFGEVFNAXXFASVYAWNRKRISNCVADYSVLYNSASFSTFKCYGVSPTKLNDLCFTNVYADSFVXXGDEVRQIAPGQTGXIADYNYKLPDDFTGCVIAWNGGSGGSGGSGGGMKVKQLEDVVEELLSVNYHLENVVARLKKLVGERGGSGGSGGSGGGNITNLCPFGEVFNAXXFASVYAWNRKRISNCVADYSVLYNSTSFSTFKCYGVSPTKLNDLCFTNVYADSFVXXGDEVRQIAPGQTGXIADYNYKLPDDFTGCVIAWN

SEQ ID NO: 31 - protein, 272 aa, Artificial Sequence
  Synthetic
  MISC_FEATURE (15)..(15): X is T or S
  MISC_FEATURE (16)..(16): X is R, T or K
  MISC_FEATURE (72)..(72): X is I or V
  MISC_FEATURE (73)..(73): X is R, T or K
  MISC_FEATURE (87)..(87): X is R, V or K
  MISC_FEATURE (180)..(180): X is T or S
  MISC_FEATURE (181)..(181): X is R, T or K
  MISC_FEATURE (237)..(237): X is I or V
  MISC_FEATURE (238)..(238): X is R, T or K
  MISC_FEATURE (252)..(252): X is R, V or K
NITNLCPFGEVFNAXXFASVYAWNRKRISNCVADYSVLYNSTSFSTFKCYGVSPTKLNDLCFTNVYADSFVXXGDEVRQIAPGQTGXIADYNYKLPDDFTGCVIAWNGGSGGSGGSGGGMKVKQLEDVVEELLSVNYHLENVVARLKKLVGERGGSGGSGGSGGGNITNLCPFGEVFNAXXFASVYAWNRKRISNCVADYSVLYNSASFSTFKCYGVSPTKLNDLCFTNVYADSFVXXGDEVRQIAPGQTGXIADYNYKLPDDFTGCVIAWN

SEQ ID NO: 32 - protein, 276 aa, Artificial Sequence
  Synthetic
MQIYEGKLTAEGLRFGIVASRANHALVDRLVEGAIDAIVRHGGREEDITLVRVCGSWEIPVAAGELARKEDIDAVIAIGVLCRGATPSFDYIASEVSKGLADLSLELRKPITFGVITADTLEQAIEAAGTCHGNKGWEAALCAIEMANLFKSLRGGSGGSGGSGGSGGGNITNLCPFGEVFNATRFASVYAWNRKRISNCVADYSVLYNSASFSTFKCYGVSPTKLNDLAFTNVYADSFVIRGDEVRQIAPGQTGKIADYNYKLPDDFTGCVIAWN

SEQ ID NO: 33 - RNA, 17 nt, Artificial Sequence
  Synthetic
gggagacugccaccaug

SEQ ID NO: 34 - RNA, 17 nt, Artificial Sequence
  Synthetic
gggagacugccaagaug

SEQ ID NO: 35 - DNA, 17 bp, Artificial Sequence
  Synthetic
taatacgactcactata

SEQ ID NO: 36 - skipped

SEQ ID NO: 37 - RNA, 17 nt, Artificial Sequence
  Synthetic
  misc_feature (13)..(13): n is c or a
  misc_feature (14)..(14): n is c or g
gggagacugccannaug

SEQ ID NO: 38 - protein, 107 aa, Artificial Sequence
  Synthetic
  MISC_FEATURE (11)..(11): X is V or I
  MISC_FEATURE (14)..(14): X is A or S
  MISC_FEATURE (15)..(15): X is T or S
  MISC_FEATURE (16)..(16): X is R, T or K
  MISC_FEATURE (24)..(24): X is N or D
  MISC_FEATURE (34)..(34): X is Y or D
  MISC_FEATURE (37)..(37): X is V or F
  MISC_FEATURE (42)..(42): X is A or T
  MISC_FEATURE (48)..(48): X is K or R
  MISC_FEATURE (61)..(61): X is C or A
  MISC_FEATURE (72)..(72): X is I or V
  MISC_FEATURE (73)..(73): X is R, T,  or K
  MISC_FEATURE (79)..(79): X is Q or E
  MISC_FEATURE (87)..(87): X is K, R, V, N or T
  MISC_FEATURE (105)..(105): X is A or S
NITNLCPFGEXFNXXXFASVYAWXRKRISNCVAXYSXLYNSXSFSTFXCYGVSPTKLNDLXFTNVYADSFVXXGDEVRXIAPGQTGXIADYNYKLPDDFTGCVIXWN

SEQ ID NO: 39 - protein, 267 aa, Artificial Sequence
  Synthetic
  MISC_FEATURE (5)..(5): X is E or C
  MISC_FEATURE (8)..(8): X is L or C
  MISC_FEATURE (9)..(9): X is T or G
  MISC_FEATURE (22)..(22): X is F or A
  MISC_FEATURE (40)..(40): X is R or C
  MISC_FEATURE (41)..(41): X is H, F or M
  MISC_FEATURE (52)..(52): X is R or C
  MISC_FEATURE (54)..(54): X is P or C
  MISC_FEATURE (61)..(61): X is V or C
  MISC_FEATURE (82)..(82): X is I or C
  MISC_FEATURE (86)..(86): X is T or C
  MISC_FEATURE (87)..(87): X is P or G
  MISC_FEATURE (88)..(88): X is H or S
  MISC_FEATURE (105)..(105): X is L or C
  MISC_FEATURE (119)..(119): X is D or C
  MISC_FEATURE (127)..(127): X is R or A
  MISC_FEATURE (131)..(131): X is K or C
  MISC_FEATURE (142)..(142): X is S or C
  MISC_FEATURE (157)..(157): optional addition of “n” repeats of (GGS)
  MISC_FEATURE (171)..(171): X is V or I
  MISC_FEATURE (174)..(174): X is A or S
  MISC_FEATURE (175)..(175): X is T or S
  MISC_FEATURE (176)..(176): X is R, T or K
  MISC_FEATURE (184)..(184): X is N or D
  MISC_FEATURE (194)..(194): X is Y or D
  MISC_FEATURE (197)..(197): X is V or F
  MISC_FEATURE (202)..(202): X is A or T
  MISC_FEATURE (208)..(208): X is K or R
  MISC_FEATURE (221)..(221): X is C or A
  MISC_FEATURE (232)..(232): X is I or V
  MISC_FEATURE (233)..(233): X is R, T or K
  MISC_FEATURE (239)..(239): X is E or Q
  MISC_FEATURE (247)..(247): X is K, R, V, N or T
  MISC_FEATURE (265)..(265): X is A or S
MQIYXGKXXAEGLRFGIVASRXNHALVDRLVEGAIDAIVXXGGREEDITLVXVXGSWEIPXAAGELARKEDIDAVIAIGVLXRGAXXXFDYIASEVSKGLADLSXELRKPITFGVITAXTLEQAIEXAGTXHGNKGWEAALXAIEMANLFKSLRGGSGGGNITNLCPFGEXFNXXXFASVYAWXRKRISNCVAXYSXLYNSXSFSTFXCYGVSPTKLNDLXFTNVYADSFVXXGDEVRXIAPGQTGXIADYNYKLPDDFTGCVIXWN

SEQ ID NO: 40 - protein, 106 aa, Artificial Sequence
  Synthetic
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAW

SEQ ID NO: 41 - protein, 106 aa, Artificial Sequence
  Synthetic
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAW

SEQ ID NO: 42 - protein, 72 aa, Artificial Sequence
  Synthetic
NSNNLDSKVGGNYNYLYRLFRKSNLKPFERDISTEIYQAGSTPCNGVEGFNCYFPLQSYGFQPTNGVGYQPY

SEQ ID NO: 43 - protein, 71 aa, Artificial Sequence
  Synthetic
NTRNIDATSTGNYNYKYRYLRHGKLRPFERDISNVPFSPDGKPCTPPALNCYWPLNDYGFYTTTGIGYQPY

SEQ ID NO: 44 - protein, 16 aa, Artificial Sequence
  Synthetic
RVVVLSFELLHAPATV

SEQ ID NO: 45 - protein, 16 aa, Artificial Sequence
  Synthetic
RVVVLSFELLNAPATV

SEQ ID NO: 46 - protein, 107 aa, Artificial Sequence; identical to SEQ ID NO: 10
  Synthetic

SEQ ID NO: 47 - protein, 107 aa, Artificial Sequence
  Synthetic
NITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWN

SEQ ID NO: 48 - protein, 66 aa, Artificial Sequence
  Synthetic
SNNLDSKVGGNYNYLYRLFRKSNLKPFERDISTEIYQAGSTPCNGVEGFNCYFPLQSYGFQPTNGV

SEQ ID NO: 49 - protein, 65 aa, Artificial Sequence
  Synthetic
TRNIDATSTGNYNYKYRYLRHGKLRPFERDISNVPFSPDGKPCTPPALNCYWPLNDYGFYTTTGI

SEQ ID NO: 50 - protein, 21 aa, Artificial Sequence
  Synthetic
GYQPYRVVVLSFELLHAPATV

SEQ ID NO: 51 - protein, 21 aa, Artificial Sequence
  Synthetic
GYQPYRVVVLSFELLNAPATV

SEQ ID NO: 52 - protein, 107 aa, Artificial Sequence; identical to SEQ ID NO: 10
  Synthetic

SEQ ID NO: 53 - protein, 107 aa, Artificial Sequence
  Synthetic
NITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWN

SEQ ID NO: 54 - protein, 107 aa, Artificial Sequence
  Synthetic
NITNLCPFGEVFNATTFASVYAWNRKRISNCVADYSVLYNSTSFSTFKCYGVSPTKLNDLCFTNVYADSFVVRGDEVRQIAPGQTGRIADYNYKLPDDFTGCVIAWN

SEQ ID NO: 55 - protein, 107 aa, Artificial Sequence
  Synthetic
NITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWN

SEQ ID NO: 56 - protein, 107 aa, Artificial Sequence; identical to SEQ ID NO: 47
  Synthetic

SEQ ID NO: 57 - protein, 107 aa, Artificial Sequence; identical to SEQ ID NO: 10
  Synthetic

SEQ ID NO: 58 - protein, 107 aa, Artificial Sequence
  Synthetic
NITNLCPFGEIFNSTRFASVYAWDRKRISNCVAYYSFLYNSASFSTFRCYGVSPTKLNDLCFTNVYADSFVIRGDEVREIAPGQTGKIADYNYKLPDDFTGCVISWN

SEQ ID NO: 59 - protein, 107 aa, Artificial Sequence; identical to SEQ ID NO: 10
  Synthetic

SEQ ID NO: 60 - protein, 107 aa, Artificial Sequence; identical to SEQ ID NO: 53
  Synthetic

SEQ ID NO: 61 - protein, 107 aa, Artificial Sequence
  Synthetic
NITNLCPFGEVFNATTFPSVYAWERKRISNCVADYSVLYNSTSFSTFKCYGVSAIKLNDLCFSNVYADSFVVKGDDVRQIAPGQTGVIADYNYKLPDDFMGCVLAWN

SEQ ID NO: 62 - protein, 107 aa, Artificial Sequence; identical to SEQ ID NO: 47
  Synthetic